(19) United States
(12) Patent Application Publication
(10) Pub. No.: US 2011/0151493 A1
(43) Pub. Date: Jun. 23, 2011

(54) POLYPEPTIDE DISULFIDE BOND ANALYSIS

(75) Inventor: Steven L. Cockrill, Longmont, CO (US)

(73) Assignee: AMGEN INC., Thousand Oaks, CA (US)

(21) Appl. No.: 12/963,460

(22) Filed: Dec. 8, 2010

Related U.S. Application Data
(60) Provisional application No. 61/294,545, filed on Jan. 13, 2010.
(60) Provisional application No. 61/285,152, filed on Dec. 9, 2009.

(51) Int. Cl. G01N 33/68; C12Q 1/37

(52) U.S. Cl. 435/23; 436/86

(57) ABSTRACT

The present invention relates in part to methods for determining bonding patterns in disulfide-linked peptides containing closely-spaced cysteine residues. Through N-terminal sequencing chemistry coupled with facile liquid chromatography and mass spectrometric analysis of the cleavage products, one can assign connectivity to specific cysteine pairs. A particular advantage of this method is maintenance of disulfide integrity during the process.

### CROSS-REFERENCE TO RELATED APPLICATIONS

This application claims the benefit which claims the benefit under 35 U.S.C. 119(e) of U.S. provisional application Ser. No. 61/285,152 filed Dec. 9, 2009, and U.S. provisional application Ser. No. 61/294,545 filed Jan. 13, 2010, the entire disclosure of which is relied upon and incorporated by reference.

### FIELD OF THE INVENTION

The present invention is generally directed to methods of analyzing polypeptides containing disulfide bonds for accurately determining connectivity of the cysteine-cysteine linkages. More particularly, the invention relates to methods for analyzing disulfide bonding patterns in the hinge region of recombinant IgG2 antibodies.

### BACKGROUND OF THE INVENTION

### Description of the Related Art

Complete elucidation of the primary structural features of a protein of interest encompasses a broad array of analytical methods to assess specific and often separate structural features, including post-translation modifications such as glycosylation, processing of terminal variants, and pairing of cysteine residues to form disulfide bonds. The disulfide linkage pattern is an important factor in determining the protein's conformational properties, and may therefore impart significant impact to the biological function through the availability of active site(s). (Flynn et al., Biochim. Biophys. Acta 1999, 1434, 177-190; Dillon et al., J. Biol. Chem. 2008, 283, 16206-16215.)

Typically, due to the large size of most proteins, cleavage of the polypeptide backbone using an appropriate endoprotease is necessary for characterization of the disulfide linkages. By comparison of the peptides under non-reducing and reducing conditions, it is possible to identify the constituent species of a disulfide-linked peptide. If these species contain only a single cysteine residue each, then elucidation of the linkage pattern is simply achieved by identification of those constituent peptides.

For species containing multiple disulfide linkages, additional cleavage between sequential cysteine residues is required; it is normally reasonable to affect cleavage between disulfide-linked cysteine residues due to the broad array of endoproteases and their associated specificities. However, the amino acid sequence of the protein of interest may contain closely-spaced cysteine residues with no suitable intermediate cleavage site, or even pairs of cysteine residues that are adjacent in the amino acid sequence. Such instances represent significant challenges to elucidation of the disulfide structure.

The contemporary literature contains several methods that may provide information for disulfide linkage analysis. Partial reduction and derivatization of the nascent free thiol group has been applied for determination of disulfide connectivity in a variety of proteins, including highly-knotted substrates (Qi et al., Biochemistry 2001, 40, 4531-4538), and those with closely-spaced cysteines (Martinez et al., Biochemistry 2008, 47, 7496-7508). However, for targeted analysis, this approach generally requires the ability to selectively reduce specific disulfide linkages. This may involve extensive optimization of the reduction conditions to afford the selective generation of isomers containing specific linkage reduction. Such optimization steps are often necessarily time-intensive and may consume significant quantities of a potentially limited sample, and are even more challenging when multiple similar linkages are present in a peptide, each with equivalent susceptibility to reduction. Additionally, the required denaturation of the protein prior to the partial reduction step may itself lead to disulfide scrambling (Yen et al., J. Mass Spectrom. 2000, 35, 990-1002). While the data output is readily interpreted, the utility of multiple steps for each cycle of processing and identification typically requires several hours of manipulation and analysis.

Automated Edman sequencing has also been utilized for determining linkage assignments through the detection of di-PTH-cystine (Wei et al., Anal. Biochem. 2002, 311, 1-9). Indeed, this approach was used to demonstrate the presence of the “ladder” structure of cysteine bonding in the hinge region of therapeutic antibodies of both the IgG4 (Wei et al., Anal. Biochem. 2002, 311, 1-9) and IgG2 (Wypych et al., J. Biol. Chem. 2008, 283, 16194-16205) subclasses. However, this technique has limitations with respect to the inability to detect of non-parallel linkages, and quantitative distinction between multiple disulfide variants present in a mixture cannot be made. Furthermore, issues with cycling efficiency and carryover are of concern, particularly with closely-spaced cysteine residues.

Recently mass spectrometry-based approaches have been applied to disulfide characterization (Wei et al., Anal. Biochem. 2002, 311, 1-9; Chelius et al., J. Am. Soc. Mass Spectrom. 2006, 17, 1590-1598; and Wu et al., Anal. Chem. 2009, 81, 112-122). Techniques employing mass spectrometric detection and analysis are very attractive options due to the fact that there is typically no need for sample preparation and manipulation beyond standard digestion and separation (i.e., peptide mapping). This approach has been successfully employed in the analysis of various protein substrates, including therapeutic antibodies (Chelius et al., J. Am. Soc. Mass Spectrom. 2006, 17, 1590-1598; and Wu et al., Anal. Chem. 2009, 81, 112-122). However, the data presented in these publications did not afford specific linkage determination between the four cysteine residues in the dimeric hinge peptide thus it was not possible to differentiate the possible parallel or “crossed” linkage patterns, i.e., bonds between Cys-229 and Cys-229, and Cys-232 and Cys-232 (parallel), or Cys-229 and Cys-232, and Cys-232 and Cys-229, “crossed”. No fragmentation was observed between the closely-spaced cysteines of each peptide chain, even with the adjacent proline residues (Wu et al., Anal. Chem. 2009, 81, 112-122).

An example application of particular interest is in the hinge region of therapeutic antibody products, particularly those of the IgG2 subclass. Determination of the disulfide structure is of importance due to increasing expectations from regulatory agencies for more complete understanding of a therapeutic molecule, but also with potential concerns over product safety and efficacy. For example, recent publications presented the identification of previously undetermined disulfide variants (Dillon et al., J. Biol. Chem. 2008, 283, 16206-16215; and Wypych et al., J. Biol. Chem. 2008, 283, 16194-16205). While novel disulfide mediated structural variants were identified, the specific linkage patterns between the Fab arm and hinge species were not determined. The MS-based methodologies described above face significant challenges in achieving elucidation of the disulfide bonding patterns of such complex structures, given the large number of cysteine residues (up to 16) present in the signature non-reduced Lys-C peptides characteristic of each structural variant (Wypych et al., J. Biol. Chem. 2008, 283, 16194-16205).

The present disclosure provides a summary of a methodology that affords unambiguous assignment of disulfide linkages in proteins/peptides containing closely-spaced or adjacent cysteine residues. The method is rapid and sensitive, and utilizes established methodologies including Edman sequencing chemistry. Data interpretation is facile, unlike contemporary methods such as MS/MS fragmentation of disulfide linked peptides. Randomization of the disulfide structure during sample processing and analysis was shown to be well controlled through optimization studies and judicious selection of reaction parameters consistent with conditions reported in current literature.

### SUMMARY OF THE INVENTION

Embodiments of the invention are directed to providing efficient and economic analysis of populations of polypeptides containing disulfide linkages. In a particular embodiment, these polypeptides are antibody or subtypes thereof. More particularly, the invention describes methods of determining connectivity between cysteine pairs by analyzing disulfide structures in IgG2. As described in further detail below, this analysis is high throughput and enables simple, rapid and accurate measurement of disulfide bonds in a population of polypeptides providing useful information for among other reasons, determining suitability of manufacturing methods, formulations, and lot release.

In accordance with the invention, provided herein is method for determining relative positions of disulfide bonds in a polypeptide. In particular, the method contemplates a method of determining the disulfide linkages in a polypeptide comprising incubating the polypeptide with an alkylating reagent, digesting the polypeptide with a protease, separating the digested non-reduced polypeptide fragments, selectively and sequentially removing the N-terminal amino acids from the non-reduced polypeptide fragments and identifying the position of the cysteine bonds.

In additional embodiments, the invention relates to a method of determining the disulfide linkages in a polypeptide comprising capping free sulfhydryls, subdividing the resulting polypeptide, separating the digested non-reduced polypeptide fragments, sequencing the fragments and identifying the position and connectivity of the cysteine bonds. In other embodiments, the invention relates to a method of determining the disulfide linkages in a polypeptide comprising denaturing the polypeptide, capping the free disulfides, subdividing the alkylated polypeptide, separating the non-reduced polypeptide fragments, subdividing the polypeptide a second time, sequencing the fragments and identifying the position of the cysteine bonds.

In further embodiments, the method utilizes an alkylating reagent, in a non-limiting example, one that is N-ethylmaleimide. In yet further embodiments the method contemplates a protease, as a non-limiting example, one that is selected from the group consisting of trypsin, Lys-C and Glu-C. In further embodiments, the method contemplates separation of the digested non-reduced polypeptide fragments by reverse phase-high performance liquid chromatography (RP-HPLC). In additional embodiments the method contemplates identification of the position of the cysteine bonds using mass spectrometry. In additional embodiments, the method contemplates that mass spectrometry is in-line with the RP-HPLC of the previous step.

In an additional embodiment, the method contemplates more than one digestion step, for example, where the method comprises determining the disulfide linkages in a polypeptide by incubating the polypeptide with an alkylating reagent, digesting the alkylated polypeptide with a protease, separating the digested non-reduced polypeptide fragments, digesting the polypeptide a second time with the same or a different protease sequencing the fragments and identifying the position of the cysteine bonds.

The invention comprises a method of determining the disulfide linkage connectivity in a polypeptide comprising incubating the polypeptide with a reagent to cap existing free sulfhydryl, dividing the polypeptide into fragments containing disulfide bonds, separating and collecting the fragments, removing sequentially the amino acid residues from the fragments, and identifying the position and connectivity of the disulfide linkages between cysteine residues. In this method, the polypeptide can be first subjected to denaturation or unfolding. Furthermore, the method can include denaturation and/or unfolding which is achieved by chemical or mechanical means. These chemical means can include but are not limited to Cyanogen bromide (CNBr), BNPS-skatole, formic acid, hydroxylamine, and 2-nitro-5-thiocyanobenzoic acid (NTCB).

In addition the invention contemplates capping free sulfhydryl with a chemical reagent. In certain embodiments, this capping is performed with a chemical reagent that is an alkylating reagent. In more particular embodiments the alkylating reagent is N-ethylmaleimide.

Further embodiments of the invention include dividing the polypeptide by chemical, enzymatic or proteolytic means. In certain embodiments, the enzyme or protease is selected from a group consisting of trypsin, Lys-C, and Glu-C. In yet further embodiments, the method contemplates use of multiple rounds of division of the polypeptide utilizing different enzymes or proteases in any given order. In yet further embodiments the invention contemplates secondarily dividing the fragments after a first dividing. In the case where proteases are used, a certain embodiment of the invention includes first proteolytically digesting the polypeptide with Lys-C, separating the fragments, then subsequently digesting the polypeptide with Glu-C.

In certain embodiments the fragments are separated by electrophoretic or chromatographic means. In these embodiments, the separation of fragments can be performed by reverse-phase high performance liquid chromatography (RP HPLC).

In yet further embodiments, the separated fragments are treated with a reagent to facilitate sequential removal of N-terminal amino acid residues. In these embodiments, the reagent can be phenylisothiocyanate (PITC). This method further includes removal of at least one N-terminal amino acid residue from the separated fragments.

In yet other embodiments, the removal of N-terminal amino acid residue(s) is achieved by acid hydrolysis. And in yet further embodiments, the acid hydrolysis is achieved using anhydrous trifluoroacetic acid (TFA). In these embodiments, the leaving and residual groups can be separated after removal of the N-terminal amino acid residues from the separated fragments. Further, the separation of leaving and residual groups can be achieved by electrophoretic or chromatographic means. In certain embodiments, the separation of leaving and residual groups can be performed by reverse-phase high performance liquid chromatography (RP HPLC).

In additional embodiments, performance of the method of the invention results leaving and residual groups whose identity can be confirmed by mass spectrometry. It is also contemplated that successive iterations of the methods described herein can be performed.

Other features and advantages of the invention will become apparent from the following detailed description. It should be understood, however, that the detailed description and the specific examples, while indicating some embodiments of the invention, are given by way of illustration only, because various changes and modifications within the spirit and scope of the invention will become apparent to those skilled in the art from this detailed description.

### BRIEF DESCRIPTION OF THE DRAWINGS

The following drawings form part of the present specification and are included to further illustrate aspects of the present invention. The invention may be better understood by reference to the drawings in combination with the detailed description of the specific embodiments presented herein.

FIG. 1 Representation of established insulin amino acid sequence and disulfide structure. Peptide annotations (A1, etc) refer to expected Glu-C peptide nomenclature for the corresponding A or B chain from the N-terminus.

FIG. 2 Assessment of insulin disulfide integrity by reverse-phase chromatography.

FIG. 3 Comparative peptide maps of insulin Glu-C digests under non-reducing and reducing conditions.

FIG. 4 EIC traces for expected A2*(s-s)B1* and potential scrambled products.

FIG. 5 Mass spectrum for doubly charged species eluting at approximately 35 minutes.

FIG. 6 Differentiation of disulfide variants through characteristic residual and leaving groups.

FIG. 7 EIC trace for A2*(s-s)B1* peptide (Structures 1, 2, and 3).

FIG. 8 EIC traces for species resulting from Structures 1, 2, and 3.

FIG. 9 EIC traces for species resulting from Structures 1 and 3.

FIG. 10 Lys-C peptide maps of nonreduced and reduced rIgG2.

FIG. 11 Fraction collection from nonreduced RP-HPLC profile of rIgG2.

FIG. 12 Non-reduced Lys-C peptide maps of rIgG2 RP-HPLC fractions.

FIG. 13 Representations of three putative structures of IgG-2A/B disulfide variants.

FIG. 14 EICs of leaving and residual groups after first cycle for IgG2-C variant.

FIG. 15 EICs of leaving and residual groups after second cycle for IgG2-C variant.

FIG. 16 Elucidated Fab-Hinge disulfide connectivity of the IgG2-C structural variant.

FIG. 17 EICs of leaving and residual groups after first cycle for IgG2-A/B variant.

FIGS. 18 A and B Elucidated Fab-hinge disulfide connectivity of the IgG2-A/B structural variants; major (IgG2-A/B1, panel A) and minor (IgG2-A/B2, second panel).

FIG. 19 EICs of leaving and residual groups after first cycle for IgG2-B variant

FIG. 20 EICs of leaving and residual groups after second cycle for IgG2-B variant.

FIGS. 21A and B Elucidated Fab-hinge disulfide connectivity of the IgG2-B structural variants; major (IgG2-B1, panel A) and minor (IgG2-B2, second panel).

### DETAILED DESCRIPTION

Structural heterogeneity of antibodies in the IgG2 subclass has been observed. For example, U.S. patent application Pub. No: 2006/194280, Dillon et al. (incorporated herein by reference in its entirety), is directed to methods of transiently enriching particular IgG isoforms by subjecting preparations of recombinant IgG proteins with a reduction/oxidation coupling reagent and optionally a chaotropic agent. The retention of these bonds is impacted by temperature, redox conditions, pH and the like and heterogeneity can reappear. Embodiments of the invention relate to rapidly and precisely analyzing disulfide bonding within a polypeptide.

Antibodies and in particular those of the IgG2 subclass have variable disulfide patterns. The observation of multiple subvariants for particular IgG2 disulfide isoforms demonstrate the degree of characterization required for full structural elucidation of a biotherapeutic product. Of interest for evaluation will be the influence of cell line and culture conditions on the distribution of the subvariant population, or potentially even the presence of alternative disulfide architecture in recombinant IgG2s, all of which is made more meaningful using the instant teachings. Certainly ongoing investigations have demonstrated the ability to exert a degree of control over the heterogeneity of IgG2 disulfide variants through site-directed mutagenesis involving Cys to Ser mutations (Allen et al., Biochemistry, 2009 48 3755-3766). At present the implication of the presence of multiple subvariants with respect to biological properties remains to be fully understood.

Insulin was also analyzed as a model protein due to its convenient size, presentation of three disulfides (two inter-chain, one intra-chain), combination of adjacent and widely-spaced cysteine residues, and a long, established history of characterization. The inter-chain disulfide linkages are between residues Cys-7 and Cys-7, and Cys-20 and Cys-19 of A chain and B chain, respectively. The intra-chain disulfide exists between Cys-6 and Cys-11 of the A chain (Chang et al., Mol. Cells, 2003, 16, 323-330).

Insulin is representative of an analytically challenging substrate that, conveniently, has been extensively characterized. Treatment of the model protein with endoprotease Glu-C following treatment with alkylating reagent under denaturing conditions simulates the typical proteolytic processing steps necessary for generation of peptides appropriate for efficient sequential release. The utility of an alkylating reagent such as, for example, N-ethylmaleimide (NEM) is employed to “scavenge” free sulfhydryl and thereby inherently prevent disulfide scrambling at the elevated temperatures and pH ranges typical of most sample preparation processes (Yen et al., J. Mass Spectrom. 2000, 35, 990-1002). Additionally, the use of lower pH buffers (i.e., pH 6.5) for digestion conditions has been utilized with specific intent of assessing disulfide linkages (Yen et al., J. Mass Spectrom. 2000, 35, 990-1002; and Foley et al., Anal. Biochem. 2008, 377, 95-104).

Literature reports also show that the expected disulfide structure of insulin may be perturbed under controlled redox conditions, resulting in scrambling of the linkages. Fortuitously, these disulfide variants are readily resolved by chromatographic means (Forsberg et al., Biochem. J. 1990, 271, 357-363) making it an exemplary polypeptide to examine using the methodology described herein.

In accordance with the above, in a particular embodiment described herein is the characterization of distinct structural isoforms of disulfide containing polypeptides including insulin and a recombinant IgG2 antibody. The information obtained from analyzing these polypeptides can then be used, for example, to adjust and perfect expression, cell culture, purification conditions, formulations and in lot release assays.

As used herein, the terms primarily forms, primarily only forms, and the like refers to antibodies that, when detected using standard methods, a substantial population exists in a single conformational isoform. Typically, a substantial population is at least 50%, at least 60%, at least 70%, at least 80%, at least 90% and more typically at least 91%, 92%, 93%, 94%, 95%, 96%, 97%, 98% or 99% of the total population.

As will be appreciated by those of skill in the art, an interchain disulfide bond is a disulfide bond between two cysteines on different polypeptides and in a particular embodiment antibody chains. Typically, an interchain disulfide bond for an antibody is between a heavy chain cysteine and a light chain cysteine or between two cysteines on different heavy chains. An intrachain disulfide bond is a disulfide bond between two cysteines on the same chain. Typically, an intrachain disulfide bond for an antibody is between a heavy chain cysteine and a heavy chain cysteine, or a disulfide bond between a light chain cysteine and a light chain cysteine on the same light chain.

In certain aspects, the light chain polypeptide always forms an interchain disulfide bond only with amino acids outside the hinge region of the heavy chain polypeptide through the most C-terminal cysteine residue of the light chain polypeptide.

### Structure of IgG2 Antibodies

IgG subtypes have predicted disulfide bond structures, particularly in their inter-heavy chain bonds and heavy chain/light chain bonds (Frangione, B. et al (1969) Nature 221, 145-148). Many differences between the several illustrated IgG isotypes can be seen in the hinge region. The IgG2 core hinge is three amino acids shorter than the core hinge of IgG1 (Dangl, J. L. et al (1988) EMBO J. 7, 1989-1994). Additionally, the IgG2 core hinge has four cysteines, one more cysteine than IgG1, and therefore has the most potential for variability in connectivity.

The hinge region has been defined in various ways (Padlan, E. A. (1994) Mol. Immun. 31, 169-217; Dangl, et al. (1988) EMBO J. 7, 1989-1994, each of which is incorporated herein by reference in its entirety). As used herein, the IgG2 hinge region includes the amino acid residues from Cys200 to Pro238, using the Eu numbering convention (Edelman, G. M. et al. (1969) Natl. Acad. Sci. U.S.A. 63:78-85, incorporated herein by reference in its entirety). Thus, an IgG2 hinge region to be analyzed according to the invention can include any modification to the portion of an IgG2 antibody that includes Eu positions 200 to 238.

### Sequencing

Any of a variety of sequencing reactions known in the art can be used to directly sequence the nucleotide sequence encoding an antibody or antibody fragment. Examples of sequencing reactions include those based on techniques developed by Maxim and Gilbert (Proc. Natl. Acad. Sci. USA, 74:560, 1977) or Sanger (Proc. Natl. Acad. Sci. USA, 74:5463, 1977). It is also contemplated that any of a variety of automated sequencing procedures can be utilized (Bio/Techniques, 19:448, 1995), including sequencing by mass spectrometry (see, e.g., PCT Publication No. WO 94/16101, Cohen et al., Adv. Chromatogr., 36:127-162, 1996, and Griffin et al., Appl. Biochem. Biotechnol., 38:147-159, 1993).

### Antigen Specificity

Embodiments of the invention are applicable to antibodies of any appropriate antigen binding specificity. Preferably, the antibodies of the invention are specific to antigens that are biologically relevant polypeptides. More preferably, the antibodies of the invention are useful for therapy or diagnosis of diseases or disorders in a mammal, such as a human.

IgG2 antibodies are particularly useful as therapeutic antibodies such as blocking antibodies, agonist antibodies, neutralizing antibodies or antibody conjugates. Non-limiting examples of therapeutic antibodies include anti-IL-1R (described in U.S. Patent Pub. No. 2004/097712), anti-RANKL (described in WO 03/002713), anti-EGFr (described in U.S. Pat. No. 6,235,883), anti-IL-4 receptor (described in U.S. Patent Pub. No. 2005/0112694 and U.S. Pat. No. 7,186,809), anti-HGF (described in U.S. Patent Pub. No. 2005/0118643), anti Ang-1 and anti-Ang-2 (described in U.S. Patent Pub. No. 2003/0124129 and WO 03/030833), anti-Ang-4, anti-OX-40, anti-GM-CSF, anti-NGF, anti-glucagon receptor, anti-sclerostin, anti-IL-17R, anti-CD30, anti-IL18, anti-activin, anti-VEGF, anti-IgE, anti-CD11, anti-CD18, anti-CD40, anti-tissue factor (TF), anti-HER2, and anti-TrkC antibodies. Antibodies directed against non-polypeptide antigens (such as tumor-associated glycolipid antigens) are also contemplated. Each of the above-mentioned references is incorporated herein by reference in its entirety.

### Production of Recombinant Antibodies

Methods for producing recombinant antibodies in mammalian cells are known. In such methods, the antibody production involves induction of protein expression. Nucleic acids encoding an IgG antibody or an IgG antibody fragment are conveniently rendered expressible by operative association with a promoter, preferably a controllable promoter functional in mammalian cells. Such recombinant constructs are designed for expression of IgG antibody protein in a suitable host (e.g., bacterial, murine, or human). Suitable promoters for expression of proteins and polypeptides herein are widely available and are well known in the art. Inducible promoters or constitutive promoters that are linked to regulatory regions (e.g., enhancers, operators, and binding regions for transcription or translation factors) are preferred. An “inducible” promoter is defined herein as a controllable promoter, including promoters typically referenced as inducible promoters (i.e., subject to positive regulation in being inactive until activated or induced by the presence of an activator or inducer) or as derepressible promoters (i.e., subject to negative regulation in being active unless a repressor is present, with removal of the repressor, or derepression, resulting in an increase in promoter activity). Promoters contemplated herein include, for example, but are not limited to, the trp, lpp, tac, and lac promoters, such as the lacUV5, from E. coli; the P10 or polyhedrin gene promoter of baculovirus/insect cell expression systems (see, e.g., U.S. Pat. Nos. 5,243,041, 5,242,687, 5,266,317, 4,745,051, and 5,169,784) and inducible promoters from other eukaryotic expression systems, as would be known in the art. For expression of the proteins, such promoters are inserted in a plasmid in operative linkage with a control region such as the operator region of the trp operon.

In addition to mammalian cells, the IgG2 molecules can be also produced in any other suitable eukaryotic or prokaryotic host cell using techniques know in the art. For example, the molecules can be produced in bacteria such as E. coli. As a further example, the molecules can be produced in insect cells transformed using baculovirus.

In some embodiments, the invention is specifically directed to improved production of IgG2 molecules. The heterogeneity of such proteins due to the presence of different disulfide bonded forms can be reduced by adjustment of processes due to information gathered by the methods described herein. These beneficial results may be assessed by monitoring such heterogeneity using the LC and LC/MS methods known to those of skill in the art.

Antibodies that retain glycosylation sites and that are secreted in mammalian cell systems will typically be glycosylated. Preferably, the proteins are secreted by mammalian production cells adapted to grow in cell culture. Examples of such cells commonly used in the industry are CHO, VERO, NSO, BK, HeLa, CV1 (including Cos), MDCK, 293, 3T3-myeloma cell lines (such as murine), PC12 and WI38 cells. Typical host cells are Chinese hamster ovary (CHO) cells, which are widely used for the production of several complex recombinant proteins, e.g. cytokines, clotting factors, and antibodies (Brasel et al., 1996, Blood 88:2004-2012; Kaufman et al., 1988, J. Biol Chem 263: 6352-6362; McKinnon et al., 1991, J Mol Endocrinol 6:231-239; Wood et al., 1990, J. Immunol 145:3011-3016). The dihydrofolate reductase (DHFR) deficient mutant cell line (Urlaub et al., 1980, Proc Natl Acad Sci USA 77:4216-4220), DXB11 and DG-44, are the CHO host cell lines of choice because the efficient DHFR selectable and amplifiable gene expression system allows high level recombinant protein expression in these cells (Kaufman R. J., 1990, Meth Enzymol 185:527-566). In addition, these cells are easy to manipulate as adherent or suspension cultures and exhibit relatively good genetic stability. CHO cells and recombinant proteins expressed in them have been extensively characterized and have been approved for use in clinical manufacturing by regulatory agencies.

Host cells are transformed or transfected with the above-described expression vectors and cultured in conventional nutrient media modified as appropriate for inducing promoters, selecting transformants, or amplifying the genes encoding the desired sequences.

Transfection refers to the taking up of an expression vector by a host cell whether or not any coding sequences are in fact expressed. Numerous methods of transfection are known to the ordinarily skilled artisan, for example, CaPO4 precipitation and electroporation. Successful transfection is generally recognized when any indication of the operation of this vector occurs within the host cell.

Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

The preparation of the IgG2 antibodies is preferably achieved in the media of the cell culture. The antibodies are produced by the cells in that culture and subsequently purified. The preparation of protein can be a cell culture supernatant, cell extract, but is preferably a partially purified fraction from the same. By partially purified means that some fractionation procedure, or procedures, have been carried out, but that more polypeptide species (at least 10%) than the desired protein or protein conformation is present. The protein can be at a fairly high concentration. Some concentration ranges are 0.1 to 20 mg/ml, more preferably from 0.5 to 15 mg/ml, and still more preferably from 1 to 10 mg/ml.

The preparation of IgG2 antibodies can be prepared initially by culturing recombinant host cells under culture conditions suitable to express the polypeptide. The polypeptide can also be expressed as a product of transgenic animals, e.g., as a component of the milk of transgenic cows, goats, pigs, or sheep which are characterized by somatic or germ cells containing a nucleotide sequence encoding the polypeptide. The resulting expressed polypeptide can then be purified, or partially purified, from such culture or component (e.g., from culture medium or cell extracts or bodily fluid) using known processes. While fractionation including but not limited to one or more steps of filtration, centrifugation, precipitation, phase separation, affinity purification, gel filtration, ion exchange chromatography, hydrophobic interaction chromatography (HIC; using such resins as phenyl ether, butyl ether, or propyl ether), HPLC, or some combination of above may be used herein, the advantageous methods of the present invention may employ LC fractionation and purification of the high molecular weight therapeutic proteins as described in U.S. patent application Pub. No: 2005/0161399, and Pub. No. 2006/194280, (each incorporated herein by reference in its entirety).

The methods described herein also may be combined with other purification methods, such as for example, purification of the polypeptide using an affinity column containing agents which will bind to the polypeptide; one or more column steps over such affinity resins as concanavalin A-agarose, heparin-Toyopearl™ or Cibacrom blue 3GA Sepharose™ one or more steps involving elution; and/or immunoaffinity chromatography. The polypeptide can be expressed in a form that facilitates purification. For example, it may be expressed as a fusion polypeptide, such as those of maltose binding polypeptide (MBP), glutathione-S-transferase (GST) or thioredoxin (TRX). Kits for expression and purification of such fusion polypeptides are commercially available from NEW ENGLAND BIOLAB® (Beverly, Mass.), PHARMACIA® (Piscataway, N.J.) and INVITROGEN®, respectively. The polypeptide can be tagged with an epitope and subsequently purified by using a specific antibody directed to such epitope. One such epitope (FLAG™) is commercially available from KODAK® (New Haven, Conn.). It is also possible to utilize an affinity column comprising a polypeptide-binding polypeptide, such as a monoclonal antibody to the protein, to affinity-purify expressed polypeptides. Other types of affinity purification steps can be a Protein A or a Protein G column, which affinity agents bind to proteins, that contain Fc domains. Polypeptides can be removed from an affinity column using conventional techniques, e.g., in a high salt elution buffer and then dialyzed into a lower salt buffer for use or by changing pH or other components depending on the affinity matrix utilized, or can be competitively removed using the naturally occurring substrate of the affinity moiety. In one embodiment of the invention, the preparation of protein may be partially purified over a Protein A affinity column.

Some or all of the foregoing purification steps, in various combinations, can also be employed to prepare an appropriate preparation of an IgG2 for use in the methods of the invention, and/or to further purify such a recombinant polypeptide after contacting the preparation of the recombinant protein with a reduction/oxidation coupling reagent. The polypeptide that is substantially free of other mammalian polypeptides is defined as an isolated polypeptide. The specific LC methods that may be combined with the redox reagent-based methods described herein are described in further detail in U.S. patent application Pub. No: 2005/0161399, and Pub. No. 2006/194280, (each incorporated herein by reference in its entirety).

The polypeptide can also be produced by known conventional chemical synthesis. Methods for constructing polypeptides by synthetic means are known to those skilled in the art. The synthetically-constructed polypeptide sequences can be glycosylated in vitro. The desired degree of final purity depends on the intended use of the polypeptide. A relatively high degree of purity is desired when the polypeptide is to be administered in vivo, for example. In such a case, the polypeptides are purified such that no polypeptide bands corresponding to other polypeptides are detectable upon analysis by SDS-polyacrylamide gel electrophoresis (SDS-PAGE). It will be recognized by one skilled in the pertinent field that multiple bands corresponding to the polypeptide can be visualized by SDS-PAGE, due to differential glycosylation, differential post-translational processing, and the like. Most preferably, the polypeptide of the invention is purified to substantial homogeneity, as indicated by a single polypeptide band upon analysis by SDS-PAGE. The polypeptide band can be visualized by fluorescent detection methods if the polypeptide incorporates an appropriate label or chromophore, silver staining, Coomassie blue staining, and/or (if the polypeptide is radiolabeled) by auto radiography.

### Characterization of Structural Isoforms of Antibodies

Determination of the conformation of a protein, and the relative proportions of a conformation of a protein in a mixture, can be done using any of a variety of analytical and/or qualitative techniques. If there is a difference in activity between the conformations of the protein, determining the relative proportion of a conformation in the mixture can be done by way of an activity assay (e.g., binding to a ligand, enzymatic activity, biological activity, etc.). Biological activity of the protein also could be used. Alternatively, the binding assays can be used in which the activity is expressed as activity units/mg of protein.

If the two conformations resolve differently during separation techniques such as chromatography, electrophoresis, filtering or other purification technique, then the relative proportion of a conformation in the mixture can be determined using such purification techniques. For example, at least two different conformations of the recombinant IgG could be resolved by way of hydrophobic interaction chromatography. Further, since far UV Circular Dichroism has been used to estimate secondary structure composition of proteins (Perczel et al., 1 991, Protein Engrg. 4:669-679), such a technique can determine whether alternative conformations of a protein are present. Still another technique used to determine conformation is fluorescence spectroscopy which can be employed to ascertain complementary differences in tertiary structure assignable to tryptophan and tyrosine fluorescence. Other techniques that can be used to determine differences in conformation and, hence, the relative proportions of a conformation, are on-line SEC to measure aggregation status, differential scanning calorimetry to measure melting transitions (Tm's) and component enthalpies, and chaotrope unfolding.

By the term isolating is meant physical separation of at least one component in a mixture away from other components it is normally associated with. For example, an antibody expressed in tissue culture is isolated from cells and media components. And an antibody expressed in the serum of an animal is isolated from the serum components it is normally associated with. Isolating components or particular conformations of a protein can be achieved using any purification method that tends to separate such components. Accordingly, one can perform multiple chromatography steps in addition to the RP-HPLC described below, including but not limited to HIC, hydroxyapatite chromatography, ion exchange chromatography, affinity, and SEC. Other purification methods are filtration (e.g., tangential flow filtration), electrophoretic techniques (e.g., electrophoresis, electroelution, isoelectric focusing), and phase separation (e.g., PEG-dextran phase separation), to name just a few. In addition, the fraction of the preparation of recombinant protein that contains the protein in the undesired conformation can be treated again in the methods of the invention, to further optimize the yields of protein with the desired conformation.

### Polypeptide Cleavage

Polypeptide cleavage can be performed by any suitable method according to the invention and examples of techniques that can be used include chemical cleavage and proteolytic cleavage. Chemical cleavage can occur using Cyanogen bromide (CNBr) which cleaves at methionine (Met) residues; BNPS-skatole which cleaves at tryptophan (Trp) residues; formic acid which cleaves at aspartic acid-proline (Asp-Pro) peptide bonds; hydroxylamine which cleaves at asparagine-glycine (Asn-Gly) peptide bonds, and 2-nitro-5-thiocyanobenzoic acid (NTCB) which cleaves at cysteine (Cys) residues. One of skill in the art will appreciate other chemical cleavage methods that are suitable for use herein.

Proteolytic cleavage can be done using proteases and can include serine proteases (e.g., trypsin, chymotrypsin, and elastase), cysteine proteases (actinidain, bromelain, calpains, caspases, cathepsins, and papain), aspartate proteases (e.g., chymosin, rennin, cathepsin D, pepsin, and plasmepsin), and metalloproteases. Particularly preferred proteases suitable for use herein include trypsin, Arg-C, Glu-C, and Lys-C. Staphylococcus aureus V-8 protease, also known as endoproteinase Glu-C, has two pH optima, one at pH 4.0 and the other at pH 7.8, and it is specific for cleavage at the carboxy terminus of glutamic acid and aspartic acid residues. Lys-C cleaves on the C-terminal side of lysine and the resulting peptides are larger than tryptic peptides.

### Capping Agents

A blocking (equivalently herein, “capping”) agent may usefully be an agent capable of alkylating, or otherwise covalently capping, free sulfhydryls. The blocking agent can thus be an alkylating agent, such as an acylhalide or alkylhalide. Examples include haloacetamides, such as iodoacetamide or iodoacetic acid; a dithiol that will form a stable disulfide bond with sulfhydryls, such as dithiobis(2-nitrobenzoic acid) (DTNB), dithiobis(5-nitropyridine); or a maleimide, such as maleimide or ethylenemaleimide. Other blocking agents include Ellman's reagent and methyl triflate. Other useful blocking agents include, for example, 4-vinylpyridine, acrylamide, dimethylacrylamide, and others.

### Activity Assays

The polypeptides of the present invention can be characterized for their physical/chemical properties and biological functions by various assays known in the art. Particularly, the quantity of an altered immunoglobulin of the present invention expressed according to a method of the invention can be compared to that of a reference immunoglobulin expressed under similar culture conditions. Methods for protein quantification are well known in the art. For example, samples of the expressed proteins can be compared for their quantitative intensities on a Coomassie-stained SDS-PAGE. Alternatively, the specific band(s) of interest (e.g., the full length band) can be detected by, for example, western blot gel analysis and/or AME5-RP assay.

Polypeptides can be further characterized by a series of assays including, but not limited to, N-terminal sequencing, amino acid analysis, non-denaturing size exclusion high pressure liquid chromatography (HPLC), cation exchange chromatography (CEX), mass spectrometry, ion exchange chromatography and trypsin digestion.

### Immunoconjugates

The invention also pertains to immunoconjugates comprising an immunoglobulin polypeptide conjugated to a cytotoxic agent such as a chemotherapeutic agent (as defined and described herein above), toxin (e.g. a small molecule toxin or an enzymatically active toxin of bacterial, fungal, plant or animal origin, including fragments and/or variants thereof), or a radioactive isotope (i.e., a radioconjugate). Conjugates of an antibody and one or more small molecule toxins, such as a calicheamicin, a maytansine (U.S. Pat. No. 5,208,020), a trichothene, and CC1065 are also contemplated herein. In one embodiment, the immunoglobulin is conjugated to one or more maytansine molecules (e.g. about 1 to). Maytansine may, for example, be converted to May-SS-Me which may be reduced to May-SH3 and reacted with modified antibody (Chari et al. Cancer Research 52: 127-131 (1992)) to generate a maytansinoid-antibody immunoconjugate.

A variety of radioactive isotopes are available for the production of radioconjugated antibodies. Examples include At²¹¹, I¹³¹, I¹²⁵, Y⁹⁰, Re¹⁸⁶, Re¹⁸⁸, Sm¹⁵³, Bi.²¹², P³² and radioactive isotopes of Lu.

Alternatively, a fusion protein comprising the immunoglobulin and cytotoxic agent may be made, e.g. by recombinant techniques or peptide synthesis.

### Pharmaceutical Formulations

Therapeutic formulations comprising an antibody are prepared for storage by mixing the antibody having the desired degree of purity with optional physiologically acceptable carriers, excipients or stabilizers (Remington's Pharmaceutical Sciences 16th edition, Osol, A. Ed. (1980)), in the form of aqueous solutions, lyophilized or other dried formulations.

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the immunoglobulin, which matrices are in the form of shaped articles, e.g., films, or microcapsule.

### Uses

An immunoglobulin may be used, for example, to purify, detect, and target a specific polypeptide it recognizes, including both in vitro and in vivo diagnostic and therapeutic methods.

In one aspect, an immunoglobulin can be used in immunoassays for qualitatively and quantitatively measuring specific antigens in biological samples. Conventional methods for detecting antigen-antibody binding includes, for example, an enzyme-linked immunosorbent assay (ELISA), a radioimmunoassay (RIA) or tissue immunohistochemistry. Many methods may use a label bound to an antibody for detection purposes.

The label used with the antibody is any detectable functionality (moiety) that does not interfere with its binding to antibody. Numerous labels are known, including the radioisotopes 32P, 32S, 14C, 125I, 3H, and 131I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, beta-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, lactoperoxidase, biotin/avidin, spin labels, bacteriophage labels, stable free radicals, imaging radionuclides (such as Technecium) and the like. Production and detection of signal associated with certain labels can be direct, or indirect involving interactions between two or more interactive moieties, such as a ligand-receptor pair, enzyme-substrate pair and fluorescence resonance energy transfer pair.

Conventional methods are available to bind these labels covalently to the immunoglobulin polypeptides. For instance, coupling agents such as dialdehydes, carbodiimides, dimaleimides, bis-imidates, bis-diazotized benzidine, and the like may be used to tag the antibodies with the above-described fluorescent, chemiluminescent, and enzyme labels. See, for example, U.S. Pat. No. 3,940,475 (fluorimetry) and U.S. Pat. No. 3,645,090 (enzymes); Hunter et al. Nature 144: 945 (1962); David et al. Biochemistry 13:1014-1021 (1974); Pain et al. J. Immunol. Methods 40:219-230 (1981); and Nygren Histochem. and Cytochem 30:407-412 (1982).

Alternative to labeling the immunoglobulin, antigen can be assayed in biological fluids by a competition immunoassay utilizing a competing antigen standard labeled with a detectable substance and an unlabeled antibody. In this assay, the biological sample, the labeled antigen standards and the antibody are combined and the amount of labeled antigen standard bound to the unlabeled antibody is determined. The amount of tested antigen in the biological sample is inversely proportional to the amount of labeled antigen standard bound to the antibody.

An immunoglobulin may be used as an affinity purification agent. In this process, the immunoglobulin polypeptide is immobilized on a solid phase such as Sephadex resin or filter paper, using methods well known in the art. The immunoglobulins can also be used as an antagonist to partially or fully block the specific antigen activity both in vitro and in vivo. Moreover, at least some of the immunoglobulins can neutralize antigen activity from other species.

In another embodiment, an immunoglobulin can be used in a method for inhibiting an antigen in a subject suffering from a disorder in which the antigen activity is detrimental, comprising administering to the subject an immunoglobulin such that the antigen activity in the subject is inhibited. Preferably, the antigen is a human protein molecule and the subject is a human subject. Alternatively, the subject can be a mammal expressing the antigen with which an antibody binds. Still further the subject can be a mammal into which the antigen has been introduced (e.g., by administration of the antigen or by expression of an antigen transgene). An immunoglobulin can be administered to a human subject for therapeutic purposes. Moreover, an immunoglobulin can be administered to a non-human mammal expressing an antigen with which the immunoglobulin cross-reacts (e.g., a primate, pig or mouse) for veterinary purposes or as an animal model of human disease.

In one aspect, a blocking antibody is specific to a ligand antigen, and inhibits the antigen activity by blocking or interfering with the ligand-receptor interaction involving the ligand antigen, thereby inhibiting the corresponding signal pathway and other molecular or cellular events. The invention also features receptor-specific antibodies which do not necessarily prevent ligand binding but interfere with receptor activation, thereby inhibiting any responses that would normally be initiated by the ligand binding. The invention also encompasses antibodies that either preferably or exclusively bind to ligand-receptor complexes. An immunoglobulin can also act as an agonist of a particular antigen receptor, thereby potentiating, enhancing or activating either all or partial activities of the ligand-mediated receptor activation.

In certain embodiments, an immunoconjugate comprising an immunoglobulin conjugated with a cytotoxic agent is administered to the patient. Preferably, the immunoconjugate and/or antigen to which it is bound is/are internalized by the cell, resulting in increased therapeutic efficacy of the immunoconjugate in killing the target cell to which it binds. Modified antibodies of the present invention can be used either alone or in combination with other compositions in a therapy. For instance, an antibody may be co-administered with another antibody, chemotherapeutic agent(s) (including cocktails of chemotherapeutic agents), other cytotoxic agent(s), anti-angiogenic agent(s), cytokines, and/or growth inhibitory agent(s).

The immunoglobulin (and adjunct therapeutic agent) is/are administered by any suitable means, including parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration.

The immunoglobulin composition of the invention will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

For the prevention or treatment of disease, the appropriate dosage of an immunoglobulin (when used alone or in combination with other agents such as chemotherapeutic agents) will depend on the type of disease to be treated, the type of antibody, the severity and course of the disease, whether the immunoglobulin is administered for preventive or therapeutic purposes, previous therapy, the patient's clinical history and response to the immunoglobulin, and the discretion of the attending physician. The immunoglobulin is suitably administered to the patient at one time or over a series of treatments. Depending on the type and severity of the disease, about 1 μg/kg to 15 mg/kg (e.g. 0.1 mg/kg-10 mg/kg) of immunoglobulin is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs.

The preferred dosage of the antibody or antibody fragment will be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, e.g. about six doses of the antibody). An initial higher loading dose, followed by one or more lower doses may be administered. An exemplary dosing regimen comprises administering an initial loading dose of about 4 mg/kg, followed by a weekly maintenance dose of about 2 mg/kg of the immunoglobulin. However, other dosage regimens may be useful. The progress of this therapy is easily monitored by conventional techniques and assays.

### EXAMPLES

The following examples are included to demonstrate some embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

The following Examples provide methods of modifying antibodies to produce structurally homogenous populations of recombinant antibody molecules.

### Example 1

### Materials

Recombinant human Insulin was sourced from Invitrogen (Carlsbad, Calif.), and insulin oxidized B-chain was from Sigma-Aldrich (St. Louis, Mo.). Endoproteinase Glu-C (protease V 8, Staphylococcus aureus V 8), sequencing grade was purchased from Roche (Indianapolis, Ind.). Reagents for manual execution of Edman sequencing including trifluoroacetic acid (TFA), phenylisothiocyanate (PITC), and N-methylpiperidine/water/methanol solution, were sequencing grade materials obtained from Applied Biosystems (Foster City, Calif.). Pyridine was ACS reagent grade from Fluka (Buchs, Switzerland). Pre-prepared mobile phases (0.1% TFA in water, and 0.1% TFA in acetonitrile), as well as HPLC-grade water, were from J. T. Baker, (Phillipsburg, N.J.).

Tris solution (1 M, pH 8.0) was from Calbiochem (La Jolla, Calif.), tris(2-carboxyethyl)phosphine (TCEP) was purchased from Pierce (Rockford, Ill.), and guanidine hydrochloride solution (8 M), iodoacetic acid (IAA), and N-ethylmaleimide (NEM) were obtained from Sigma-Aldrich (St. Louis, Mo.). NAP-5 Sephadex G-25 columns were sourced from Pharmacia Biotech (Little Chalfont, UK).

### Chromatographic Analysis of Commercial Human Insulin

Commercial human insulin was assessed by reverse-phase HPLC in order to ascertain the potential presence of multiple disulfide structures. A 1 μg load of insulin (1 mg/mL in 2 M Gdn HCl, 100 mM Tris, pH 6.5) was applied to a reverse phase column (Agilent Zorbax 300SB C8, 2.0×150 mm, 5 μm particle size) and separated using mobile phases consisting of A): 20% acetonitrile in 200 mM ammonium sulfate and 50 mM sulfuric acid; and B): 40% acetonitrile in 200 mM ammonium sulfate and 50 mM sulfuric acid. Elution was accomplished using a 30 minute gradient from 10% to 50% mobile phase B at a temperature of 40° C. and a flow rate of 0.2 mL/min, as reported previously (Hua et al., Biochemistry, 2002, 41, 14700-14715).

Enzymatic Digestion with Endoprotease Glu-C and Mapping of Insulin Peptides

Insulin was reconstituted in 400 μL of denaturation buffer (6M Guanidine HCl, 200 mM Tris, pH 6.5) and then treated with 100 μL HPLC grade water and 12 μL of 0.5 M iodoacetic acid for a final protein concentration of 2 mg/mL. The sample was incubated at room temperature in the dark for 30 minutes, then buffer exchanged to 50 mM Tris, pH 6.5 using an equilibrated NAP-5 Sephadex G-25 column, with a resulting protein concentration of 1 mg/mL.

Digestion using endoprotease Glu-C was accomplished by incubating 120 μL of 1 mg/mL insulin solution (in 50 mM Tris buffer, pH 6.5) with Glu-C (substrate to enzyme ratio 3:1) overnight at room temperature in foil-wrapped sample tubes. Digestion was quenched by addition of 12 μL of 10% TFA in water.

Separation of the resulting peptides was accomplished using a 1200 HPLC system (Agilent, Santa Clara, Calif.) equipped with a binary pump. A reverse-phase column (Agilent Zorbax 300SB C8, 2.1×150 mm, 5 μm particle size) was employed with mobile phases consisting of (A) 0.1% TFA in water and (B) 0.1% TFA in acetonitrile. A linear gradient between 2-50% mobile phase B was used, following a 5 minute system equilibration step at 2% B, with a flow rate of 0.2 mL/min. Reduced mapping was performed under identical conditions following treatment of the digest products with 2 μL tris(2-carboxyethyl)phosphine (TCEP) for 10 minutes at room temperature.

For LC-MS analysis, the outlet of the HPLC separation was coupled to a MSD-TOF ESI-MS instrument (Agilent, Santa Clara, Calif.) running in positive ion mode. Source settings were a scan range of 200-3000 m/z, fragmentor, skimmer, octopole RF, and capillary voltages of 200 V, 60 V, 250 V, and 4,000 V, respectively. Drying gas was supplied at 10.0 L/min and 300° C. Nebulizer pressure was 40 psig. For fraction collection, the MS instrument was bypassed, and the HPLC eluent collected manually and dried by vacuum centrifugation prior to further processing and analysis.

### Coupling Reaction

Derivatization of primary amines was accomplished using phenylsothiocyanate (PITC), as traditional Edman degradation chemistry (Tarr, Anal. Biochem. 1975, 63, 361-370). Briefly, the peptides resulting from Glu-C digestion of 4 nmol of insulin and isolated by peptide mapping as described above were reconstituted in 10 μL of 10 mM N-ethylmaleimide NEM in water, then incubated at room temperature in the dark for 30 minutes. A 40 μL volume of anhydrous pyridine, 5 μL of PITC, and a 5 μL volume of N-methylpiperidine/water/methanol solution were added under dry nitrogen. Coupling was achieved by incubation at various temperatures (room temperature, 50° C.) for various times (3 to 20 minutes). The solution was then dried completely by vacuum centrifugation. Completeness of coupling was assessed by resolubilization in 30% pyridine in water and analysis by LC-MS, using separation and detection conditions as described for execution of the peptide map.

### Cleavage Reaction

Cleavage of the derivatized substrate was accomplished by acid treatment using anhydrous TFA. Various modes were assessed, including direct addition of a 10 μL aliquot of liquid TFA to the dried sample under dry nitrogen, and in TFA vapor (introduced by a filter paper soaked in 20 μL of anhydrous TFA), and incubating at 50° C. or room temperature for 2 to 10 minutes. Completeness of cleavage was assessed by LC-MS, using the conditions noted above.

### Conversion Reaction

The solution resulting from the cleavage step detailed above was cooled and pooled by centrifuging for 2 minutes at room temperature. A 15 μL aliquot of HPLC-grade water was added to reach a final TFA concentration of 25%, after which the solution was incubated at 64° C. for 10 minutes. After cooling, a portion of the samples was loaded directly onto the LC-MS system for analysis. Note that these conditions were designed to be identical to those of the automated Procise 494 instrument (Applied Biosystems, Foster City, Calif.).

### Iterative Coupling/Cleavage Cycles

For sequential cycles, the residual substrate following a given alkylation/coupling/cleavage/conversion cycle was collected from the chromatographic separation, dried by vacuum centrifugation, and then subjected to repetition of the alkylation, coupling, cleavage, and conversion steps followed by LC-MS analysis as described above.

### Results

### Assessment of Insulin Disulfide Integrity

The disulfide integrity of recombinant human insulin used as a model protein was assessed chromatographically. As reported previously (Hua et al., Biochemistry, 2002, 41, 14700-14715), scrambled products may chromatographically resolved from the native structure using a reverse phase separation. The recombinant insulin showed the presence of a single peak. Due to the absence of multiple peaks in the reverse phase profile, it is inferred that the model protein utilized for method development presented the expected disulfide arrangement.

Isolation of Disulfide-Linked Peptides from Recombinant Human Insulin

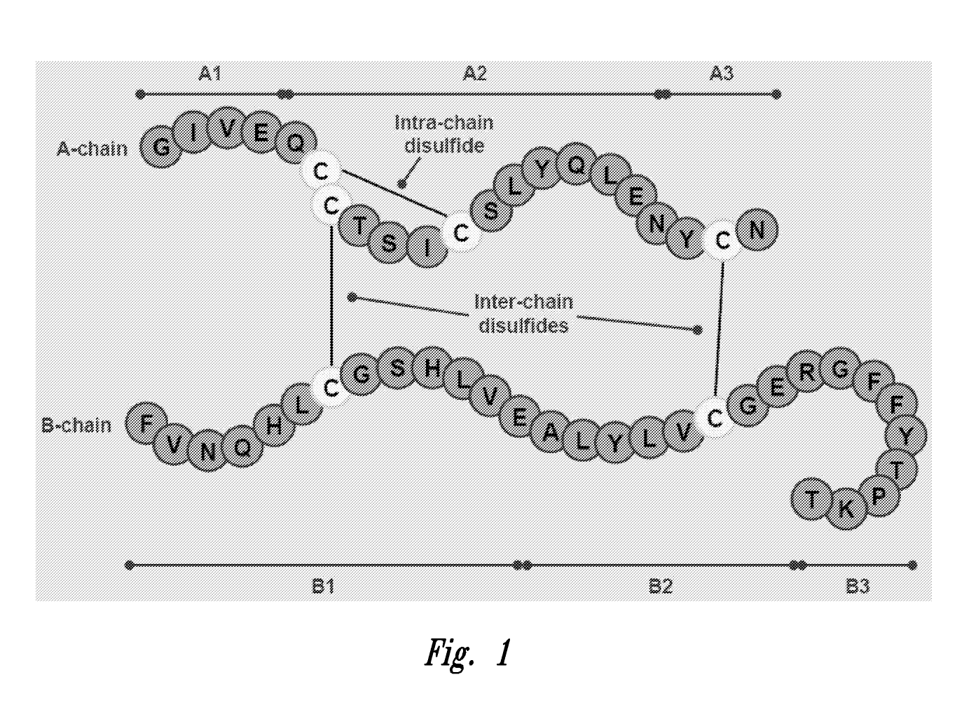

Insulin contains three disulfide linkages between its six cysteine residues. Digestion with endoprotease Glu-C under non-reducing conditions putatively generates four proteolysis products, summarized in Table 1. Comparative peptide mapping of the digest under non-reducing and reducing conditions (FIG. 1) affords identification of peaks corresponding to disulfide-containing peptides through either retention time shift, mass difference, or both. Two peptides were identified as containing disulfide linkages—peaks eluting at ˜29 minutes and ˜36 minutes, as they were absent from the chromatogram upon treatment with reducing agent. The peak eluting at ˜29 minutes was tentatively identified as peptide A3(s-s)B2 by comparison of its observed and theoretical masses (1,376.58 Da and 1,376.59 Da, respectively—Table 1). Similarly, the peak eluting at ˜36 minutes was identified as peptide A2(s-s)B1 (2,967.32 Da and 2,967.33 Da for observed and theoretical masses, respectively—Table 1)

TABLE 1
Putative cleavage products of insulin digestion by
endoprotease Glu-C under non-reducing conditions

| | Structure/ | Theoretical | Observed | Mass Error |
|---|---|---|---|---|
| Peptide | Identity | Mass (Da) | Mass (Da) | (ppm) |
| A1 | GIVE | 416.23 | 416.23 | 0 |
| A2(s-s) | QCCTSICSLYQLE(s-s) | 2,967.33 | 2,967.32 | 3 |
| B1 | FVNQHLCGSHLVE | | | |
| A2 | QCCTSICSLYQLE | 1,489.63 | 1,489.63 | 0 |
| B1 | FVNQHLCGSHLVE | 1,481.71 | 1,481.72 | 7 |
| A3(s-s) | NYCN(s-s)ALYLVCGE | 1,376.59 | 1,376.58 | 7 |
| B2 | | | | |
| A3 | NYCN | 512.17 | 512.17 | 0 |
| B2 | ALYLVCGE | 866.42 | 866.42 | 0 |
| B3 | RGFFYTPKT | 1,115.58 | 1,115.58 | 0 |

Note
masses are monoisotopic

The disulfide connectivity of non-reduced peptides containing a single disulfide bond may be determined directly from confirmation of the constituent species. Peak A3(s-s)B2 eluting at ˜29 minutes yielded two product peaks upon treatment with reducing agent (peaks A3 and B2 in FIG. 1), and these species were identified by mass spectrometry as NYCN and ALYLVCGE (observed masses of 512.17 Da and 866.42 Da, respectively—Table 1). As the identified peptides contain only a single Cys residue each, the disulfide linkage of peptide A3(s-s)B2 must be through the two cysteine residues—i.e., Cys-20 in peptide A3 is bound to Cys-19 in peptide B2.

Peptide A2(s-s)B1 eluting at ˜36 minutes also yielded two product peaks (peptides A2 and B1 in FIG. 1). Mass spectrometry detection of the product peaks confirmed their identities as QCCTSICSLYQLE and FVNQHLCGSHLVE (observed masses of 1,489.63 Da and 1,481.72 Da, respectively—Table 1) through matching of the expected and observed masses. Due to the fact that one of peptides contains three cysteine residues, confirmation of the specific linkage pattern cannot be inferred from the constituent peptides, nor can the presence of co-eluting disulfide variants. In order to ascertain the connectivity of the disulfide linkages in peptide A2(s-s)B1, a new methodology was developed, as described in the following sections.

### MANUAL EDMAN OF GLU-C PEPTIDE A2(S-S)B1

### Principle of the Method

The methodology comprises a convergence of traditional Edman chemistry executed manually with analysis using LC-MS. Employment of extracted ion chromatograms is a key aspect, on account of the fact that the UV trace is confounded by remnant reagent peaks. The mechanisms for Edman chemistry have been extensively documented (Tarr, Anal. Biochem. 1975, 63, 361-370; Creighton, Proteins: Structure and Molecular Properties, 2nd ed.; W. H. Freeman and Co.: New York, 1993). Under basic conditions, the phenylisothiocyanate couples with alpha amino groups (i.e., the N-terminus) to form a phenylthiocarbamyl (PTC) group. This is cleaved by treatment with anhydrous acid, releasing the anilinothiazolinone (ATZ) derivative of the N-terminal amino acid and generating a new N-terminus for repetitive cycles. Conversion of the ATZ derivative to the more stable phenythiohydantoin (PTH) derivative results in the loss of water, and a consequent reduction in mass.

### Optimization Studies of Manual Edman Reaction

Optimization studies were accomplished using a simpler test molecule—Insulin oxidized B-chain. Coupling and cleavage steps are summarized below.

### Coupling Reaction (Insulin Oxidized B Chain)

Incorporation of the PITC label on primary amines was assessed by comparing 2% vs. 20% of the total reaction volume at 50° C. for 20 minutes. Insulin oxidized B-chain contains two primary amines—the free N-terminus and a lysine residue at position 29. Evaluation of extracted ion chromatograms for the unlabeled, singly-, and doubly-labeled peptide demonstrated that under the coupling conditions, both 2% and 20% proportions generate essentially complete labeling at primary amines (data not shown). Therefore, in order to accommodate more complex disulfide-linked peptides containing more primary amines, an intermediate value of 10% PITC was selected.

A time-course study to evaluate the labeling requirements using 10% PITC and 50° C. reaction temperature was performed. Reaction times of 3, 5, and 20 minutes were assessed. In each case, 100% label incorporation (i.e., doubly-labeled peptide) was observed—however at 20 minutes, a side reaction that generated a +12 Da adduct mass was observed. Therefore 5 minutes was selected as the optimal reaction time.

Incubation temperature was also probed. Automated Edman sequencing programs typically employ 50° C. reaction temperatures, however, in order to mitigate potential thermally-induced disulfide rearrangements, a comparison of incubation at 50° C. and room temperature was employed for insulin oxidized B-chain, and also angiotensin II as a secondary test peptide. The results indicated that room temperature incubation was sufficient to yield complete labeling of primary amines (data not shown).

Automated programs for Edman sequencing employ washing steps with highly hydrophobic solvents (ethyl acetate and heptane) to remove the small molecule by-products of the coupling reaction. However, it has been reported in the literature that exposure to organic solvents could potentially lead to disulfide scrambling (Chang et al., J. Biol. Chem. 2001, 276, 9705-9712). In order to assess the impact on the peptide detectability of the washing step, the coupling reaction was performed using insulin oxidized B-chain, with and without the washing step. The results demonstrated that although the reagent peaks were diminished with washing, recovery of the coupled peptide was reduced by approximately 50% with the washing step (peak areas of 2,973,728 vs. 1,385,883 by summed EIC of multiple charge states or 1,368.78 vs. 675.20 by UV, for exclusion or inclusion of the wash step, respectively—data not shown). The issues with compromised recovery as well as the potential for scrambling of disulfide-linkages was considered to be of sufficient risk to the value of the method that the washing step was omitted from the final protocol.

Therefore, the optimized coupling conditions were determined to be addition of a 40 μA volume of anhydrous pyridine, 5 μA of PITC (10% of the total reagent volume), and 5 μL of methyl-piperidine under dry nitrogen to the reconstituted target peptide. Coupling was achieved by incubation at room temperature for 5 minutes, after which the solution containing coupled peptide was dried completely by vacuum centrifugation. Note that coupling was performed after reconstitution of the target peptide in 10 μA of 10 mM NEM in water and incubation at room temperature in the dark for 30 minutes in order to mitigate disulfide scrambling. Evaluation of the final coupling conditions on insulin Glu-C peptide A2(s-s)B1 showed that the expected incorporation of two PITC labels was essentially 100% complete.

### Cleavage Reaction

Removal of the N-terminal amino acid residue following the coupling step was accomplished by acid cleavage, based upon the conditions used with automated instrumentation programs. Cleavage was found to be complete after incubating with 10 μL neat anhydrous TFA for 2 minutes at 50° C., i.e., no partial cleavage products were observed by LC-MS. The utility of room temperature incubation was found to be not sufficient for complete cleavage (data not shown). Vapor phase sequencing reactions have been reported in the literature to afford higher sensitivity (Brandt et al., Anal. Biochem. 1988, 168, 314-323), however in our assessment there was no discernable advantage using vapor phase TFA over liquid TFA, most likely due to the fact that the remaining steps of the degradation chemistry are performed in solution phase. Liquid phase TFA was employed in the final method on account of the fact that subsequent manipulations were more readily facilitated. The completeness of the final cleavage reaction conditions was also demonstrated using insulin peptide A2(s-s)B1 (data not shown).

### Conversion

In automated Edman, a conversion step from the AZT derivative to the more stable PTH derivative is performed. This was accomplished simply by monitoring the masses of the resulting products—converted (PTH) and unconverted (AZT) differ by the loss of water, and are therefore readily differentiated by MS detection. The conversion step is performed under identical conditions to that established for automated N-terminal sequencing using a Procise 494 instrument (Applied Biosystems, Foster City, Calif.). Conversion was found to be 100% complete after incubating with 25% TFA at 64° C. for 10 min, and therefore was not optimized further.

### Confirmation of Disulfide Integrity During Execution of Optimized Technique

A major concern with any methodology employed to probe disulfide bonding is the occurrence of linkage scrambling or rearrangement. Therefore an assessment of scrambling potential was made by performing the coupling, cleavage, and conversion steps in the presence of NEM. By incorporating the alkylating reagent during the processing steps, any rearrangement of the disulfides would be captured through the labeling of the transitional free sulfhydryl, and the resulting formation of unique products. Five distinct products would indicate the occurrence of scrambling—peptide B1* with a single NEM label, peptide A2* with a single NEM label, peptide A2* with three NEM labels, B1* dimer, or A2* dimer (where the * notation indicates the loss of the first N-terminal residue from the annotated peptide). Each of these species has a unique mass, and likely a characteristic retention time.

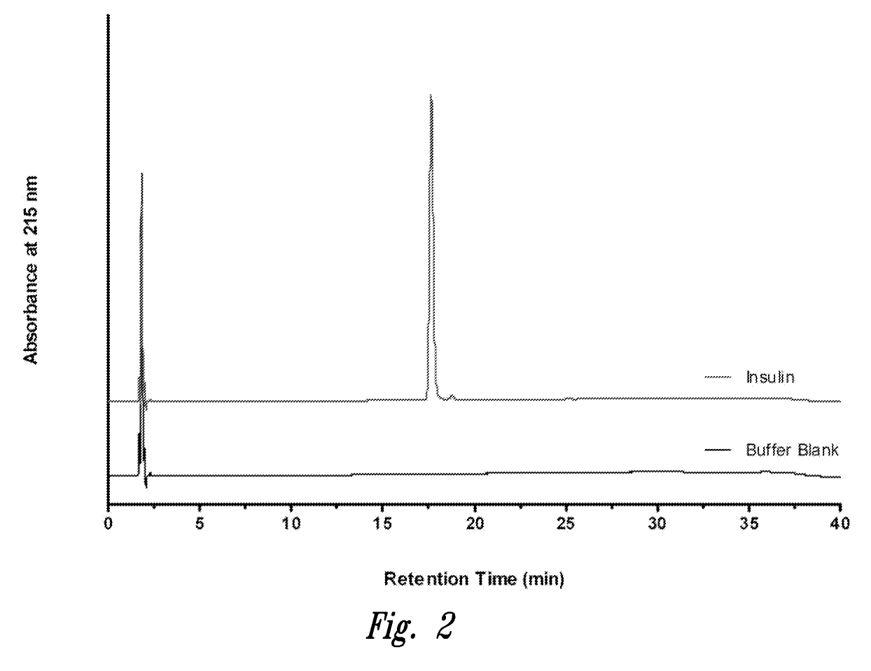

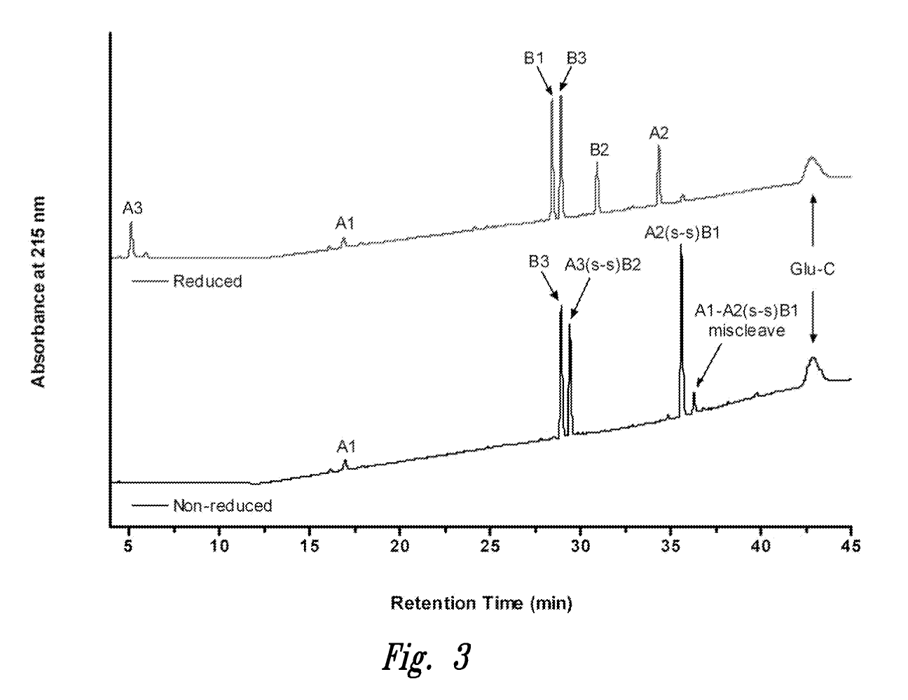

Insulin peptide A2(s-s)B1 was pretreated with 10 mM NEM at room temperature for 30 minutes before execution of the manual Edman coupling, cleavage, and conversion cycles in order to identify the propensity for disulfide scrambling. FIG. 2 shows the EIC of possible NEM derivatives of peptides A2* and B1*, the A2* dimer and B1* dimer species, as well as of the expected disulfide-linked structure of peptide A2*(s-s)B1*. The fact that no detectable quantities of the potential scrambled products were observed indicates that the disulfide structure was not compromised by execution of the coupling, cleavage, and conversion steps of the Edman reaction. The small signal in the A2* dimer (theoretical mass 2,717.13 Da) EIC trace is related to the sodium adduct of the expected A2*(s-s)B1* structure (theoretical mass 2,692.21 Da). The fourth isotope of the [M+H+Na]²⁺ ion of the expected A2*(s-s)B1* structure exhibits the same m/z value as the first isotope of the [M+2H]²⁺ ion of the A2* dimer (FIG. 3). The fact that the two signals occur at the same elution time suggests that they are related to the same structure (i.e., protonated vs. sodiated), which is considerably more likely than co-elution of different structures. The absence of B1* dimer also supports this proposal.

Theoretical Distinction of Disulfide Variants for Glu-C Peptide A2(s-s)B1

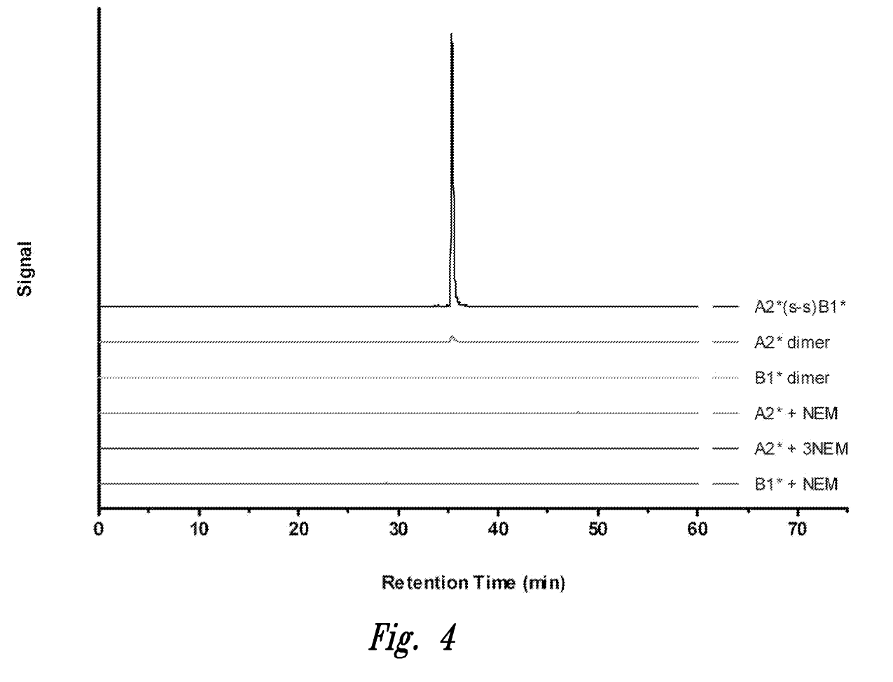

Three putative structures (1, 2 and 3) of peptide A2(s-s)B1 resulting from the Glu-C digestion of insulin are presented in FIG. 4, which differ in the connectivity but all share the same theoretical mass of 2,967.33 Da. In structure 1 the disulfide linkages are Cys-6 to Cys-11 (intra-chain on Chain A), Cys-A7 to Cys-B7 (inter-chain). This represents the traditionally accepted linkage pattern (Chang et al., Mol. Cells, 2003, 16, 323-330; Hua et al., Biochemistry, 2002, 41, 14700-14715). The other two structures contain variations of the disulfide linkages; structure 2 contains and inter-chain linkage between Cys-6 of the A-chain to Cys-7 of the B-chain, and an intra-chain linkage between Cys-7 and Cys-11 of the A-chain. Structure 3 contains an intra-chain linkage between Cys-6 and Cys-7 of the A-chain, and an inter-chain linkage between Cys-11 of the A-chain and Cys-7 of the B-chain. Note that these represent purely hypothetical arrangements given the confirmed constituents of peptide A2(s-s)B1, although both structures 2 and 3 have been generated under conditions of limited reduction and denaturation (Hua et al., Biochemistry, 2002, 41, 14700-14715).

The three species are differentiated by their respective residual and leaving groups following sequential cycles of Edman sequencing chemistry, as demonstrated in FIG. 4. The first cycle does not permit distinction as each of the species simply loses the glutamine and valine residues from A- and B-chains, respectively. While the products of structures 1 and 3 remain isobaric after the second cycle, structure 2 is clearly differentiated as two product species are formed of masses 1,256.56 and 1,471.59 Da. Three cycles are necessary to distinguish structures 1 and 3, after which the A- and B-chains of structure 1 are cleaved, resulting in the formation of two product species of mass 1,391.57 and 1,357.55 Da, respectively. Structure 3 is not cleaved into separate chains by three cycles of Edman chemistry, and as such the residual mass is 2,275.08 Da.

Application of Repetitive Cycles to Insulin Peptide A2(s-s)B1

As noted in FIG. 4, the presence of potential disulfide variants may be distinguished only after at least two cycles, due to the location of the first cysteine at the second amino acid position in peptide A2. Therefore multiple rounds of coupling, cleavage, and conversion steps were executed in sequential manner.

### Cycle 1

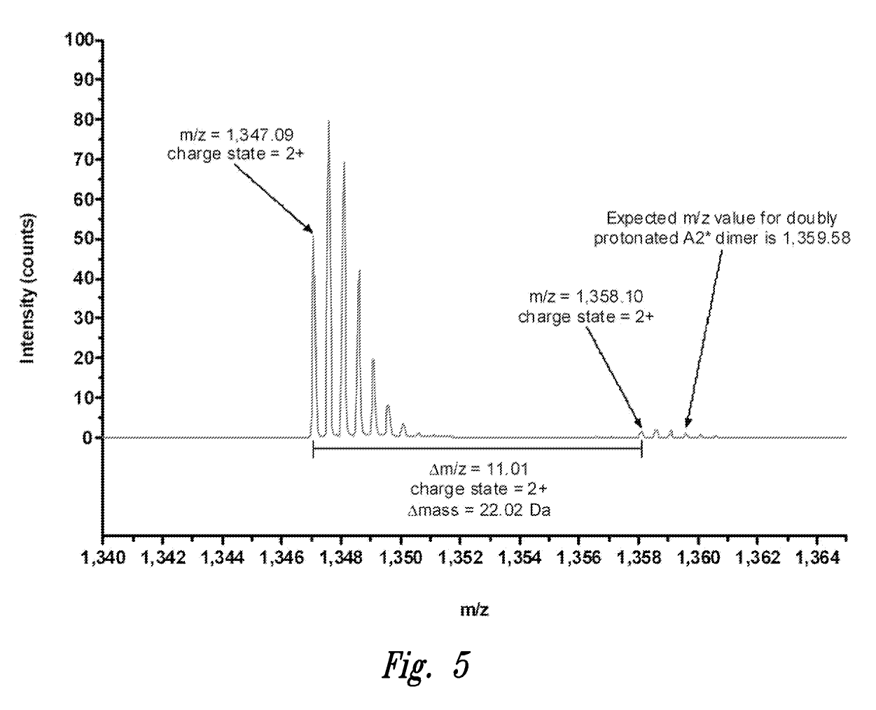

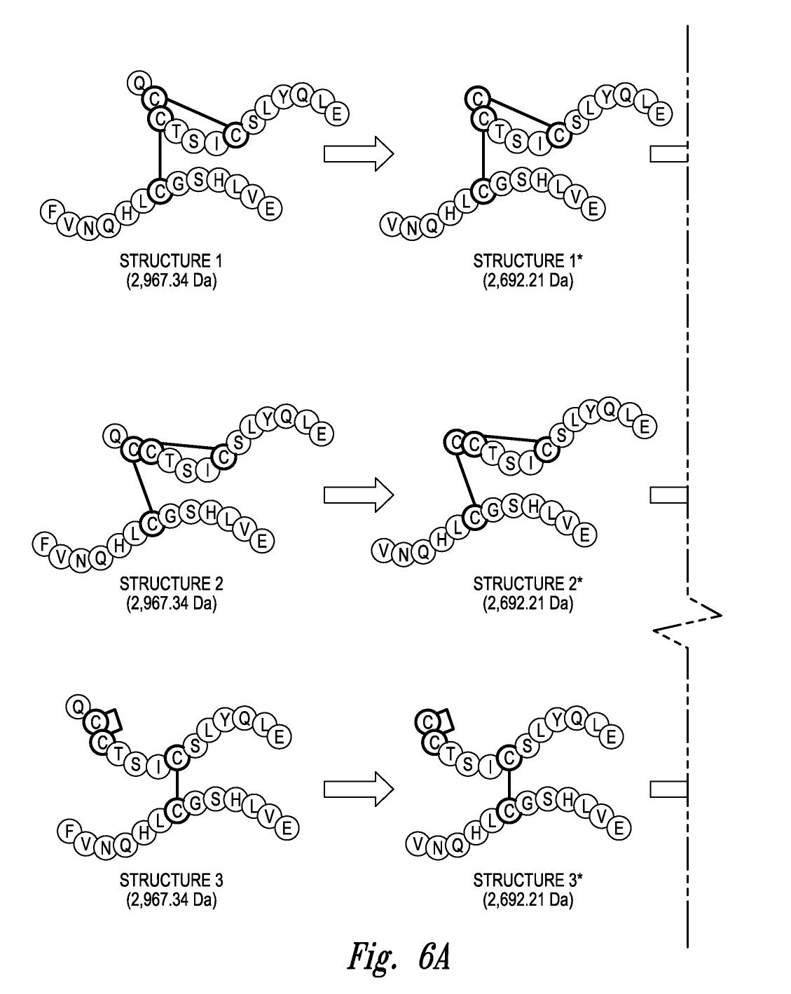

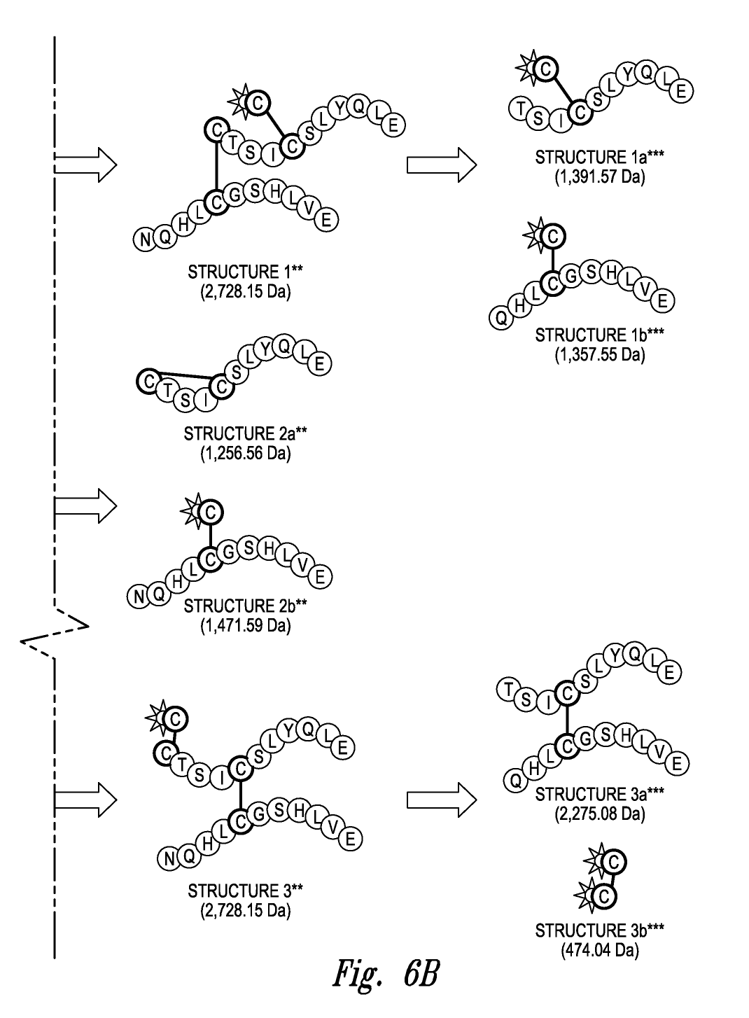

The first cycle of N-terminal truncation does not result in distinguishing species as each of the three structures 1*, 2*, and 3* possess the same mass. The EIC for peptide A2*(s-s)B1 is shown in FIG. 5. These data indicate that the expected species, A2*(s-s)B1* was detected. As noted above (FIG. 2), potential scrambling products were not detected.

### Cycle 2

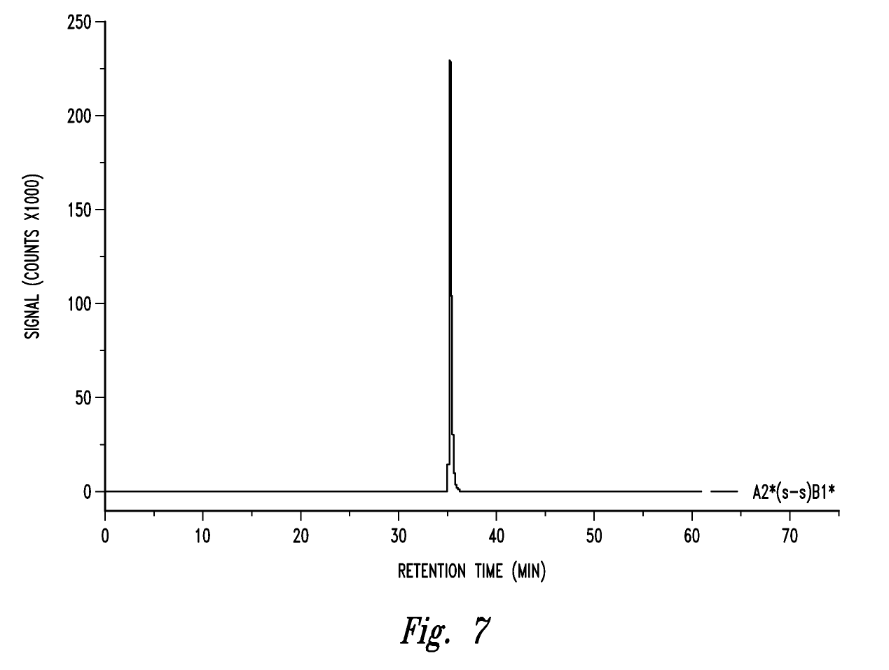

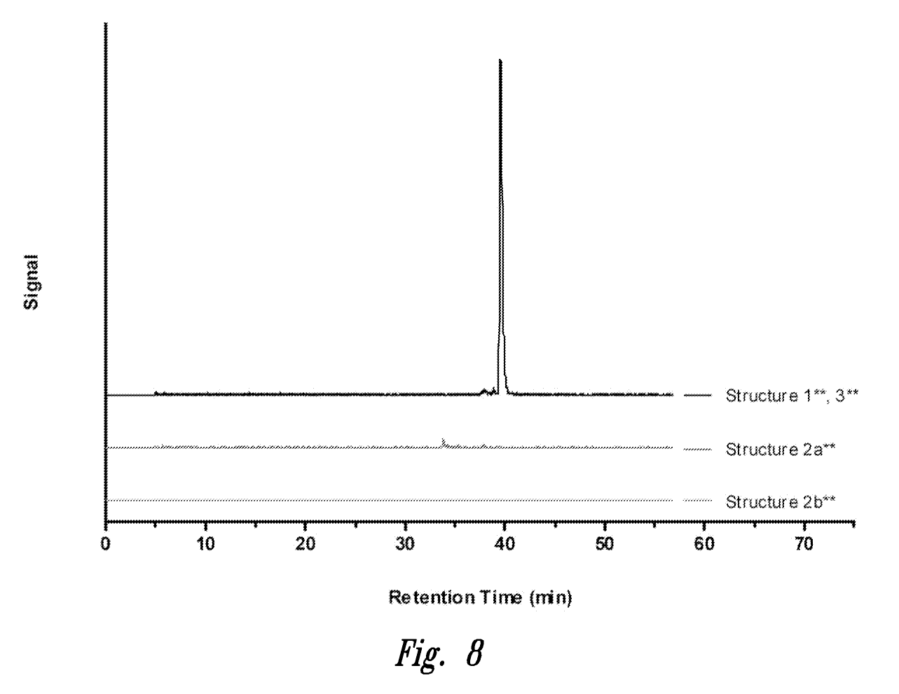

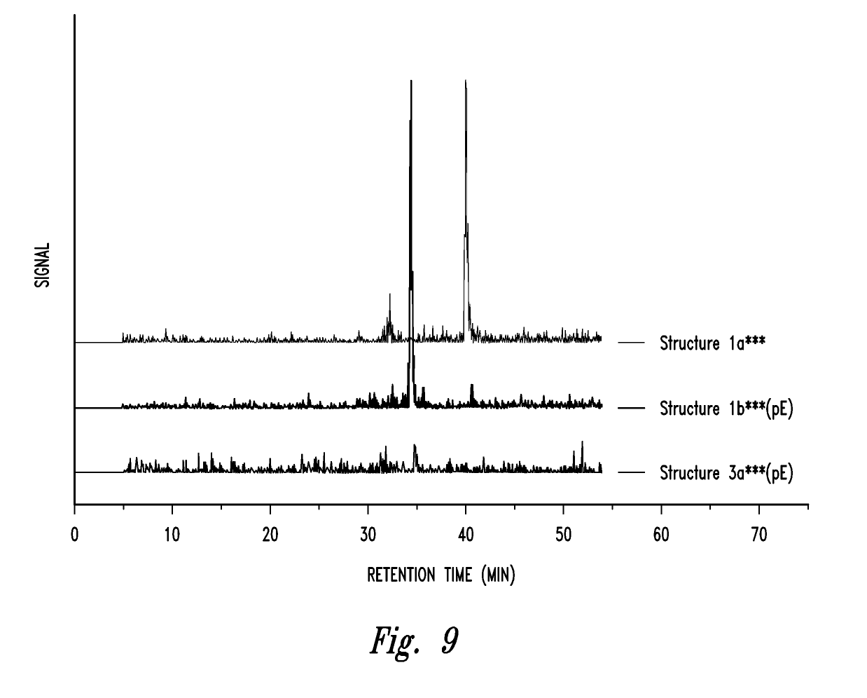

The second cycle of N-terminal truncation is the first opportunity at which the discrete disulfide variant structures may be differentiated. As noted above, Structures 1** and 3** (i.e., A2**(s-s)B1**), where ** indicates the completion of two Edman cycles, cannot be resolved by mass at this point, as they still possess the same constituent amino acid inventory. However, structures 2a** (peptide A2**) and 2b** (peptide B1**) are readily differentiated from structures 1** and 3**. EICs for these structures are presented in FIG. 6. The traces demonstrate that the dominant species identified has a mass that corresponds to the expected structure (Structure 1**), although clearly no distinction can be made regarding the presence of structure 3**. What is evident is the fact that no detectable levels of the species corresponding to structure 2 are observed, therefore it is concluded that this particular unexpected disulfide variant is not present in the population of insulin molecules.

### Cycle 3

The third cycle of N-terminal truncation provides differentiation of Structures 1 and 3. After three cycles, Structure 1 dissociates to discrete peptides from each chain (structure 1a*** and 1b*** are peptides A2*** and B1*** respectively). Completion of three cycles for Structure 3 results in the loss of a di-PTH-cystine group (structure 3b***), and a residual group that contains peptides A2*** and B1*** bound through a disulfide linkage. EICs for these structures are presented in FIG. 7. Note that the di-PTH-cystine is not reliably detected due to its small mass or poor retention on the column.

The traces demonstrate that the dominant species identified are structures 1a*** and 1b***, which corresponds to the expected structure (Structure 1). Note that structure 1b*** and 3a*** were detected as the pyroglutamylate derivative (with concomitant loss of 17.03 Da), as the N-terminal residue of each species is a glutamine. Very minor amounts of species derived from Structure 3 were detected, again demonstrating that the native disulfide structure is essentially preserved throughout multiple rounds of manual Edman cycling.

One aspect to note is the reduction in signal to noise through sequential steps (FIG. 5, FIG. 6, and FIG. 7), which is recognized as a limitation of the present methodology. This is analogous to the limited cycling efficiency of automated Edman degradation, which typically demonstrates a repetitive yield of ˜90% (Miyashita et al., Proc. Natl. Acad. Sci. U.S.A. 2001, 98, 4403-4408), however, it is apparent that the losses in the manual methodology may be higher due to the need to recover the residual groups from multiple chromatographic steps.

Improvements in yield and sensitivity may be afforded through coupling the separation with a nanospray ionization source capable of simultaneous fraction collection, such as the Advion NanoMate™ Triversa. Preliminary data in our laboratory suggests that this may afford significant sensitivity improvements due to the properties of the nanospray interface, as well as throughput gains by eliminating the need for separate chromatographic steps for analysis and collection.

### Example 2

### Materials

The recombinant human IgG2 monoclonal antibody was expressed with κ-type light chains and γ2-type heavy chains. The antibody was produced from Chinese hamster ovary (CHO) cell culture and purified using established techniques (Shukla et al., J. Chromatogr. B. 2007 848, 28-39).

Endoprotease Lys-C was sourced from Wako Chemicals (Richmond Va.), and endoproteinase Glu-C (sequencing grade) was purchased from Roche (Indianapolis, Ind.).

Reagents for manual execution of Edman sequencing including trifluoroacetic acid (TFA), phenylisothiocyanate (PITC), and N-methylpiperidine/water/methanol solution, were sequencing grade materials obtained from Applied Biosystems (Foster City, Calif.). Pyridine was ACS reagent grade from Fluka (Buchs, Switzerland). Pre-prepared mobile phases (0.1% TFA in water, and 0.1% TFA in acetonitrile), as well as HPLC-grade water, n-propanol, were from J. T. Baker, (Phillipsburg, N.J.). Tris was from Calbiochem (La Jolla, Calif.); tris(2-carboxyethyl)phosphine (TCEP) and trifluoroacetic acid (TFA) for mobile phase preparation were purchased from Pierce (Rockford, Ill.). Sodium acetate, urea, hydroxylamine hydrochloride, guanidine hydrochloride solution (8 M), and N-ethylmaleimide (NEM) were obtained from Sigma-Aldrich (St. Louis, Mo.).

### Separation of Disulfide Variants

Disulfide variants were isolated using a non-reduced reverse-phase high performance liquid chromatography (RP-HPLC) method using an Agilent 1100 system, based on previous reports (Wypych et al., J. Biol. Chem. 2008, 283, 16194-16205; Dillon et al., J. Biol. Chem. 2008, 283, 16206-16215; and Dillon et al., J. Chromatogr. A. 2006 1120 112-120). Mobile phases consisted of A) 0.11% TFA in 0.39% n-propanol, 0.11% acetonitrile, and B) 0.10% TFA in 70% n-propanol, 20% acetonitrile. Maximal loading of an analytical scale RP-HPLC column (Agilent Zorbax SB300 C18, 4.6×250 mm, 5 μm particle size) without resolution loss was employed, and elution accomplished with a linear gradient between 24-29% mobile phase B in 25 minutes after a 5 minute equilibration at 15% mobile phase B. Flow rate was 0.5 mL/min, and detection monitored at 215 nm. Each isoform was enriched from multiple rounds of fraction collection and pooling. Fractions were dried by vacuum centrifugation and stored at −20° C. until analysis. Mass analysis was also performed by coupling an ESI-TOF instrument (Applied Biosystems Q-STAR Pulsar I, Foster City, Calif.) to the chromatographic instrument. Deconvolution of the charge envelope across the isoform profile was accomplished using Analyst QS software (v.2.0, Applied Biosystems).

### Non-Reduced Peptide Mapping of Unfractionated and Enriched Disulfide Variants

A 150 μL volume containing 900 μg of rIgG2 was denatured using 350 μL of denaturation buffer (8 M guanidine HCl, 10 mM N-ethylmaleimide (NEM), 0.1 M sodium acetate, pH 5.2) and incubated for 3 hours at 37° C. NEM was present to alkylate free sulfhydryl and mitigate the potential for disulfide scrambling (Bures et al., Biochemistry 37, 12172-12177). The entire 500 μL volume was added to 20 mL of Lys-C digestion buffer (4 M urea, 20 mM hydroxylamine, 0.1 M Tris, pH 7.0) and treated with 450 μL of endoprotease Lys-C (2 mg/mL in water). Proteolysis was accomplished by incubation at 37° C. overnight, after which the digestion was quenched by addition of 250 μL of 5% trifluoroacetic acid (TFA). Samples for assessment of the reduced peptide map were then subjected to reduction by incubation with 5 μL of Tris(2-carboxyethyl)phosphine hydrochloride (TCEP) at room temperature for 30 min.

Enriched disulfide variants were processed with different volumes of reagent, depending upon their proportion in the non-reduced RP-HPLC separation (RP-1 and RP-3 were treated with higher volumes; RP-2, RP-4, and RP-5 were treated with lower volumes). Each fraction was reconstituted in 10 or 20 μL of denaturation buffer, denatured by incubation at 37° C. for 3 hours, then treated with 400 or 800 μL of Lys-C digestion buffer. Each fraction was treated with 9 or 18 μL of Lys-C solution and digested overnight at 37° C., after which the digest was quenched by addition of 5 or 10 μL of 5% TFA.

Separation was accomplished using an Agilent 1200 HPLC instrument using a reverse-phase column (Vydac 214TP52 C4, 2.1×250 mm) thermostatted to 60° C. Mobile phases were A) 0.1% TFA in water, B) 0.1% TFA in 90% acetonitrile. Digested samples (˜150 μg) were injected, and separated using a linear gradient of 0-45% mobile phase B in 90 minutes after a 10 minute equilibration step. A flow rate of 200 μL/min was employed, and peptide elution monitored by absorbance at 215 nm and on-line mass spectrometry using an ESI-TOF instrument (Agilent MSD-TOF, Santa Clara, Calif.) in positive ion mode. Fraction collection of disulfide-linked peptides for further characterization was accomplished via by-passing the mass spectrometer inlet and using an automated fraction collector (Agilent). Isolated peptides were dried by vacuum centrifugation and stored at −20° C. until analysis.

### Secondary Digestion of Disulfide-Linked Peptides

Peptides with multiple constituent peptides and two or more disulfide linkages were subjected to secondary digestion with endoprotease Glu-C. Peptide G (from the classical structure IgG2-A) was reconstituted in 100 μL of Glu-C digestion buffer (2 M guanidine HCl, 100 mM Tris, pH 6.5) and incubated with 5 μL of endoprotease Glu-C (1 mg/mL in digestion buffer) overnight at room temperature. The digest products were then analyzed by LC-MS to confirm identity of the resulting peptide species. Separation was accomplished by HPLC (Agilent 1200, Santa Clara, Calif.) using a reverse-phase column (Agilent Zorbax 300SB C8, 2.1×150 mm, 5 μm particle size) thermostatted to 50° C. Mobile phases were A) 0.1% TFA in water, B) 0.1% TFA in acetonitrile. Samples after secondary digestions were injected, and separated using a linear gradient of 2-50% mobile phase B in 45 minutes after a 5 minute equilibration step. A flow rate of 200 μL/min was employed, and peptide elution monitored by absorbance at 215 nm and on-line mass spectrometry using an ESI-TOF instrument (Agilent MSD-TOF, Santa Clara, Calif.) in positive ion mode. Manual fraction collection was performed under identical conditions, except that the mass spectrometer inlet was bypassed.

Disulfide-linked peptides b, c, and f (containing species representative of the different variants IgG2-C, IgG2-A/B, and IgG2-B, respectively) were collected from non-reduced Lys-C peptide maps generated from digestion of 10 mg of unfractionated rIgG2. Each collected peptide pool was dried completely by vacuum centrifugation and reconstituted in 400 μL of Glu-C digestion buffer. Digestion was performed by addition of 20 μL of endoprotease Glu-C (1 mg/mL in digestion buffer) and carried out overnight at room temperature. The digest products were then analyzed by LC-MS to confirm identity and by LC with fraction collection of the relevant pools containing disulfide-linked peptides unique to a specific variant. Separation and detection parameters were as summarized above.

### Automated N-Terminal Sequencing

N-terminal sequencing using automated Edman degradation chemistry was performed on the hinge:hinge dipeptide (Peptide F). Briefly, peptide F was collected from the non-reduced Lys-C peptide map of unfractionated rIgG2, and diluted with 0.1% TFA to reduce the organic solvent content to below 15%. The purified peptide was applied to a ProSorb PVDF membrane pretreated with 10 μL of methanol. Samples were loaded into the sample cartridge and analyzed on an automated sequencer (Applied Biosystems Procise 494, Foster City, Calif.) using the Pulsed Liquid PVDF program designed for samples adsorbed onto PVDF membranes.

### Manual N-Terminal Sequencing

Manual coupling and cleavage of amino acid residues from disulfide-linked species characteristic to each variant was performed according to an optimized protocol derived in-house using traditional Edman chemistry (Zhang and Cockrill, Anal. Chem. 2009). Briefly, each disulfide-linked peptide fraction after digestion with endoproteases Lys-C and Glu-C was collected and dried by vacuum centrifugation. The sample was resuspended in 10 μL of 10 mM NEM, and incubated in the dark at room temperature for 30 min in order to mitigate potential disulfide scrambling. The sample was then treated with 40 μL of anhydrous pyridine, 5 μL of phenylisothiocyanate (PITC) and 5 μL of N-methylpiperidine/water/methanol solution and incubated at 50° C. for 3 min under dry nitrogen. The sample was then dried by vacuum centrifugation and cleavage of N-terminal residues performed by resuspending the sample in 5 μL TFA and incubating at 50° C. for 2 min under a nitrogen atmosphere. The cleaved sample was then resuspended in 40 μL of 4 M guanidine HCl, 100 mM Tris, pH 6.5. The leaving and residual groups were identified by loading the complete 40 μL volume on by capillary scale RP-HPLC coupled to a linear ion trap ESI instrument (Thermo LTQ XL, Waltham, Mass.). Separation was performed using an Agilent 1200 HPLC instrument using a reverse-phase column (Agilent Zorbax 300SB C8, 1.0×150 mm, 3 μm particle size) thermostatted to 50° C. Mobile phases were A) 0.1% TFA in water, B) 0.1% TFA in acetonitrile. Samples after secondary digestions were injected, and separated using a linear gradient of 2-71% mobile phase B in 65 minutes after a 5 minute equilibration step. A flow rate of 50 μL/min was employed, and peptide elution monitored by absorbance at 215 nm and on-line mass spectrometry.

Data from the LC-MS analysis was parsed for specific mass values corresponding to potential residual and leaving groups, depending on the known inventory of peptides associated with each unique peptide. A second identical preparation of each sample was employed to collect the residual group(s) for additional processing cycles. Iterative coupling and cleavage cycles were executed by repeating the steps noted above.

### Identification of Disulfide-Linked Peptides by Peptide Mapping

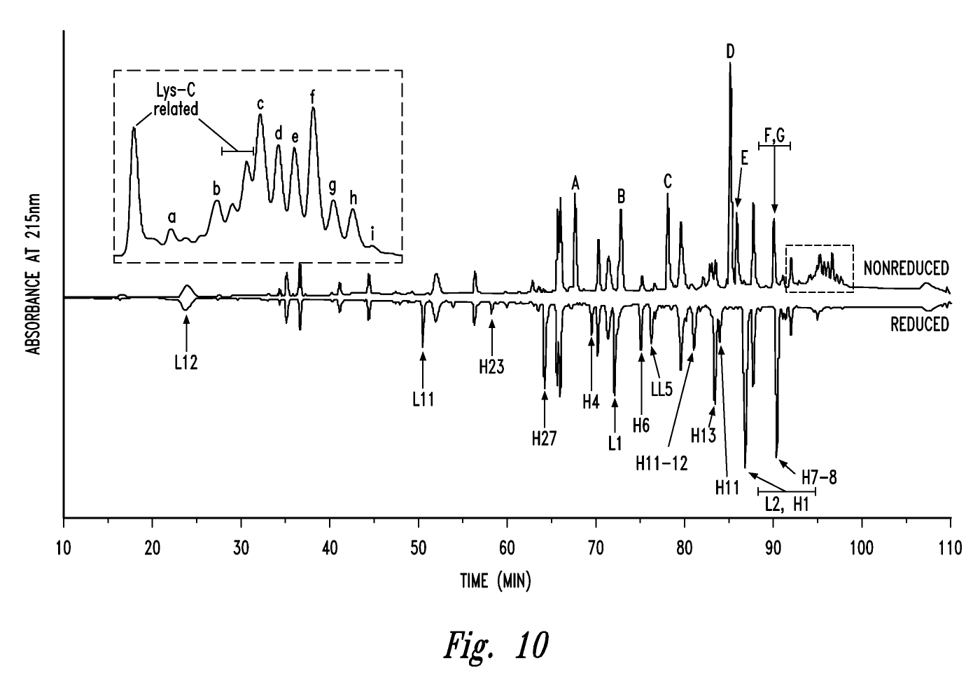

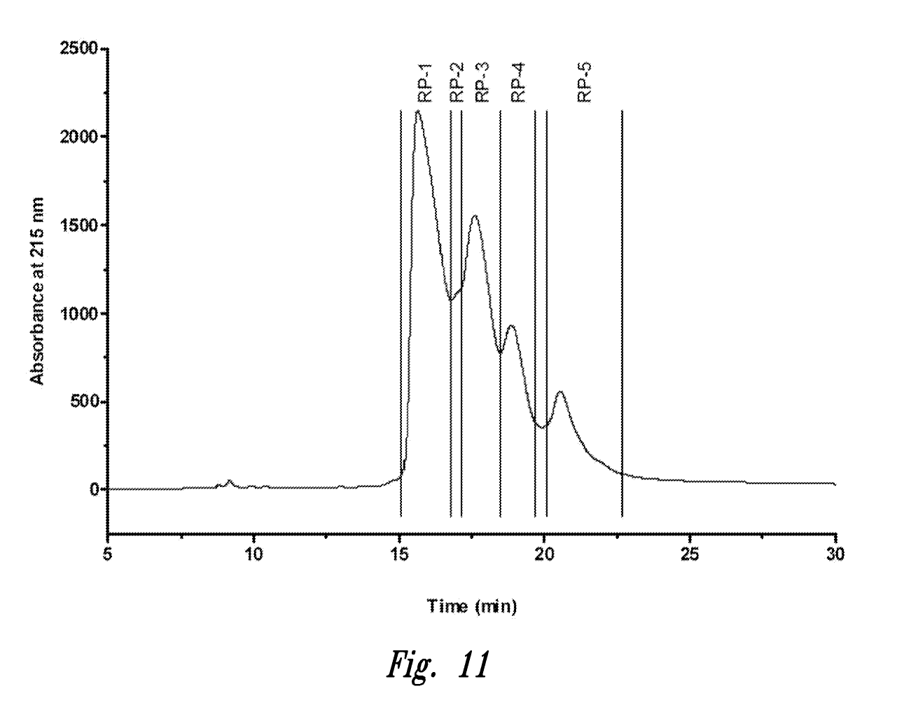

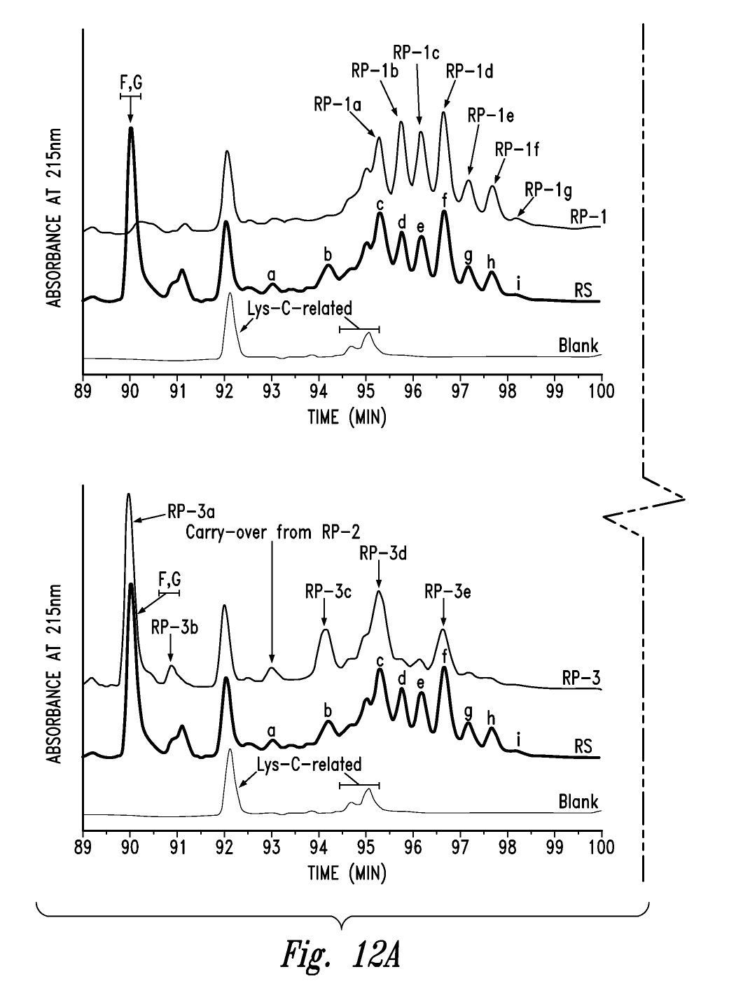

Comparison of peptide maps of unfractionated IgG2 following digestion with endoprotease Lys-C under non-reduced and reduced conditions provided determination of peptides bound through disulfide-linkages by their appearance in the non-reduced map and absence following reduction. The peptide maps are presented in FIG. 10. Peptides F and G co-eluted in the non-reduced Lys-C peptide map (FIG. 10) and hence and additional separation was performed using an alternative reverse-phase column (Phenomenex Jupiter C4, data not shown). The non-reduced map shows peaks A through G which are absent upon reduction. Disappearance of these peptides after reduction confirms that they are involved in disulfide linkages in the native protein. The highlighted region of FIG. 10 indicates additional species that also disappear upon reduction. These are disulfide-linked peptides corresponding to variants of the IgG2 disulfide structure, and their characterization is discussed later.

### Determination of Connectivity for Peptides A Through G

Comparison of theoretical to measured mass values confirmed the identities of the constituent species from the disulfide-bound peptides. Non-reduced peptides containing a single disulfide bond allows determination of the disulfide connectivity directly from mass confirmation of the constituent species. The observed masses of peptides A through G, and their product peptides after treatment with reductant, are summarized in Table 2.

Peptide F showed the generation of two product peaks upon treatment with reducing agent. Mass spectrometry detection of the two product peaks confirmed their identities as H11 and H11-12 through matching of the expected and observed masses. Note that peptide H11-12 contains a missed cleavage at a Lys-Pro sequence. Due to the fact that peptide H11 contains four closely-spaced cysteine residues, confirmation of the specific linkage pattern cannot be inferred from the constituent peptides.

The conserved amino acid sequence of IgG2 heavy chain indicates that the dimeric hinge:hinge peptide (Peptide F) contains eight cysteine residues, and therefore four disulfide linkages. Both the amino acid sequence and the closely-spaced cysteines impose limitations as to the degree of characterization that can be performed. However, if the disulfide bonding is present as a “ladder” in which each cysteine is bound to its counterpart on the other hinge peptide, then Edman sequencing will yield the detection of di-PTH-Cys which has been reported to elute near the position of PTH-Tyr (Zhang et al., Anal Biochem 2002 311 1-9). Previous characterization of the dimeric hinge:hinge peptide by Edman degradation suggested linkage consistent with literature reports of four parallel disulfides per the classical IgG2 structure. Hence, peptide F was subjected to automated N-terminal sequencing analysis, the results of which demonstrated the detection of di-PTH-Cys moieties in each of cycles 1, 2, 5, and 8 (data not shown).

The automated N-terminal sequencing data confirms the presence of a parallel disulfide linkage structure in the dimeric hinge:hinge peptide (peptide F), in which each cysteine in one chain is bound to the corresponding residue in the other chain. This is consistent with the classical IgG2-A structure, and as reported in the literature (Wypych et al., J. Biol. Chem. 2008, 283, 16194-16205; Zhang et al., Anal Biochem 2002 311 1-9). However, the presence of alternate connectivity cannot be ruled out, due to the quantitative limitations of the N-terminal sequencing method.

TABLE 2
Constituent Species of Expected Disulfide-linked Peptides

| | Constituent | Observed | Theoretical | Mass Error |
|---|---|---|---|---|
| Peptide | Peptides | Mass (Da) ^{a} | Mass (Da) ^{a} | (ppm) |
| A | (Intact) | 4090.55 | 4090.61 | 14.67 |
| | H23 | 1104.32 | 1104.33 | 9.42 |
| | H27 | 2988.35 | 2988.29 | 19.84 |
| B | (Intact) | 3886.36 | 3886.39 | 7.72 |
| | L5 | 2069.34 | 2069.35 | 3.09 |
| | L11 | 1819.03 | 1819.05 | 13.30 |
| C | (Intact) | 4806.29 | 4806.35 | 12.48 |
| | H13 | 4559.04 | 4559.03 | 2.19 |
| | H17 | ND | 249.33 | ND |
| D | (Intact) | 11286.54 | 11286.50 | 3.54 |
| | Ll | 4439.02 | 4439.00 | 4.37 |
| | L2 | 6849.34 | 6849.51 | 24.75 |
| E | (Intact) | 6996.83 | 6996.88 | 7.15 |
| | H1 | 4430.91 | 4430.99 | 17.74 |
| | H4 | 2567.85 | 2567.90 | 19.59 |
| F | (Intact) | 5129.13 | 5129.21 | 15.60 |
| | H11-12 | 2681.24 | 2681.28 | 14.92 |
| | H11 | 2455.94 | 2455.99 | 20.36 |
| G | (Intact) | 10096.14 | 10096.13 | 0.99 |
| | H6 | 2578.99 | 2578.97 | 7.76 |
| | H7-8 | 6709.30 | 6709.32 | 2.98 |
| | L12 | 811.87 | 811.87 | 0.00 |

^{a} Average masses reported

Peptide G showed the generation of three product peaks upon treatment with reducing agent. Mass spectrometry detection of the product peaks confirmed their identities as H6, H7-8, and L12 through matching of the expected and observed masses. The constituent peptides contain a total of four cysteine residues; peptide H6 contains two cysteines (Cys-136 and Cys-149); accordingly there are two possible structural variants of the disulfide bonding for peptide G. Fortuitously, the presence of a glutamic acid residue at position 142 of peptide H6 afforded a cleavage site between the two cysteine residues with endoprotease Glu-C, resulting in formation of H6a (N-terminal portion) and H6b (C-terminal portion).

Following Glu-C digestion and subsequent analysis by LC-MS the formation of two product peptides was revealed. Mass analysis of the secondary digest products demonstrated that the two product peptides were 2444.65 Da and 7669.47 Da. The former mass is consistent with species in which the N-terminal portion of peptide H6 (H6a, containing Cys-136) remains bound to L12 through Cys-215. No cleavage occurs at Glu-214 in L12, likely as a result of steric hindrance from the disulfide bond, and no cleavage occurs at Glu-251 in L7-8 on account of the two flanking proline residues. The peptide of mass 7669.47 Da corresponds to the C-terminal portion of H6 (H6b, containing Cys-149) after cleavage at Glu-142, coupled to peptide H7-8 through Cys-205. Hence, the elucidated disulfide bonding for peptide G is consistent with the classical structure previously described (Wypych et al., J. Biol. Chem. 2008, 283, 16194-16205). Note that digest products of the alternative structure were not observed, indicating that a single homogeneous bonding pattern exists for the Fab arm intra-chain linkage in the IgG2 molecule. In addition, this also indicates that disulfide exchange is not observed during sample processing.

Taken together, characterization of the disulfide-linked peptides A through G elucidated the linkages between specific Cys residues, summarized in Table 3, which confirm the presence of the expected classical disulfide structure (IgG2-A).

TABLE 3
Confirmed Connectivity for Nonreduced Peptides A through G

| | Constituent | | | |
|---|---|---|---|---|
| Peptide | Peptides | Cys-Cys Bonds Identified | Loop | Element |
| A | (H23)/(H27) | Cys-368 (H23)-Cys-426 | C_{H}3 | Fc |
| | | (H27) | | |
| B | (L5)/(L11) | Cys-135 (L5)-Cys-195 (L11) | C_{L} | Fab |
| C | (H13)/(H17) | Cys-262 (H13)-Cys-322 | C_{H}2 | Fc |
| | | (H17) | | |
| D | (L1)/(L2) | Cys-23 (L1)-Cys-89 (L2) | V_{L} | Fab |
| E | (H1)/(H4) | Cys-22 (H1)-Cys-96 (H4) | V_{H} | Fab |
| F | (H11-12)/ | Cys-224 (H11)-Cys-224 | — | Hinge |
| | (H11) | (H11) | | |
| | | Cys-225 (H11)-Cys-225 | | |
| | | (H11) | | |
| | | Cys-228 (H11)-Cys-228 | | |
| | | (H11) | | |
| | | Cys-231 (H11)-Cys-231 | | |
| | | (H11) | | |
| G | (H6)/(H7- | Cys-149 (H6)-Cys-205 (H7-8) | C_{H}1 | Fc |
| | 8)/(L12) | Cys-136 (H6)-Cys-215 (L12) | | |

### Characterization of Non-Reduced Peptides Unique to Disulfide Variants

In addition to characterization of non-reduced peptides A through G which confirmed the classical IgG2 disulfide structure, the non-reduced Lys-C peptide map also showed the presence of other species eluting between 90 and 100 min (peaks a through i) that disappeared following treatment with reducing agent (FIG. 10). The observed masses and putative constituent species of these non-reduced peptides are summarized in Table 4.

TABLE 4
Peptides Corresponding to IgG2 Disulfide Variants

| | Observed | Theoretical | Constituent Disulfide |
|---|---|---|---|
| Peak | Mass (Da) ^{a} | Mass (Da) ^{a} | Bound Peptides |
| a | 12773.34 | 12773.38 | (H11-12)/(H6)/(H7-8)/(L12) |
| b | 15450.88 | 15450.63 | (H11-12)2/(H6)/(H7-8)/(L12) |
| | 12548.07 | 12548.09 | (H11)/(H6)/(H7-8)/(L12) |
| c | 15225.49 | 15525.34 | (H11)/(H11-12)/(H6)/(H7-8)/(L12) |
| | 26161.04 | 26160.46 | (H10-11-12)/(H11-12)/(H6)2/ |
| | | | (H7-8)2/(L12)2 |
| d | 25547.28 | 25546.75 | (H11-12)2/(H6)2/(H7-8)2/(L12)2 |
| e | 25935.81 | 25935.18 | (H10-11-12)/(H11)/(H6)2/(H7-8) |
| | | | 2/(L12)2 |
| f | 25321.98 | 25321.47 | (H11-12)/(H11)/(H6)2/(H7-8) |
| | | | 2/(L12)2 |
| | 15000.19 | 15000.05 | (H11)2/(H6)/(H7-8)/(L12) |
| g | 25710.40 | 25709.89 | (H11)/(H10-11)/(H6)2/(H7-8) |
| | | | 2/(L12)2 |
| | 24794.35 | 24793.89 | (H11)/(H11-12)/(H6)2/(H7)/ |
| | | | (H7-8)/(L12)2 |
| h | 25096.83 | 25096.18 | (H11)2/(H6)2/(H7-8)2/(L12)2 |
| i | 24568.52 | 24568.60 | (H11)2/(H7)/(H7-8)/(H6)2/(L12)2 |

^{a} Average masses reported

Three distinct groupings of characteristic masses (˜12 kDa, ˜15 kDa, and ˜25 kDa) are observed in the mass spectrometric data for peaks a through i (Table 4). Multiple non-reduced peptides corresponding to each grouping are observed; for example, peaks b, c, and f contain peptides with masses of 15450.88, 15225.49, and 15000.19 Da, respectively, all of which are related to the ˜15 kDa grouping. The mass difference between these peptides is consistent with the presence of 0, 1, or 2 copies of the H12 peptide through variable cleavage at the Lys-Pro sequence in H11-12. Given that observed mass distribution for each grouping is the result of partial cleavage it was postulated that the related species of each grouping share a common disulfide structure, and therefore characterizations of peaks containing representative species for each grouping was performed.

The three groupings of non-reduced Lys-C peptides correspond to disulfide variants, determined based upon the constituent masses of each grouping. The ˜12 kDa variant contains a single copy of the hinge peptide (H11) connected to a single copy of each of the Fab arm peptides (H6, H7, and L12). The ˜15 kDa variant contains two copies of the hinge peptide connected to a single copy of each of the Fab arm peptides. Finally, the ˜25 kDa variant contains two copies of the hinge peptide connected to two copies of each of the Fab arm peptides. Two of these are consistent with the previously reported variants termed IgG2-A/B (˜15 kDa variant), IgG2-B (˜25 kDa variant) (Wypych et al., J. Biol. Chem. 2008, 283, 16194-16205). The ˜12 kDa (IgG2-C) variant was noted in the prior account.

### Isolation and Enrichment of Disulfide Variants

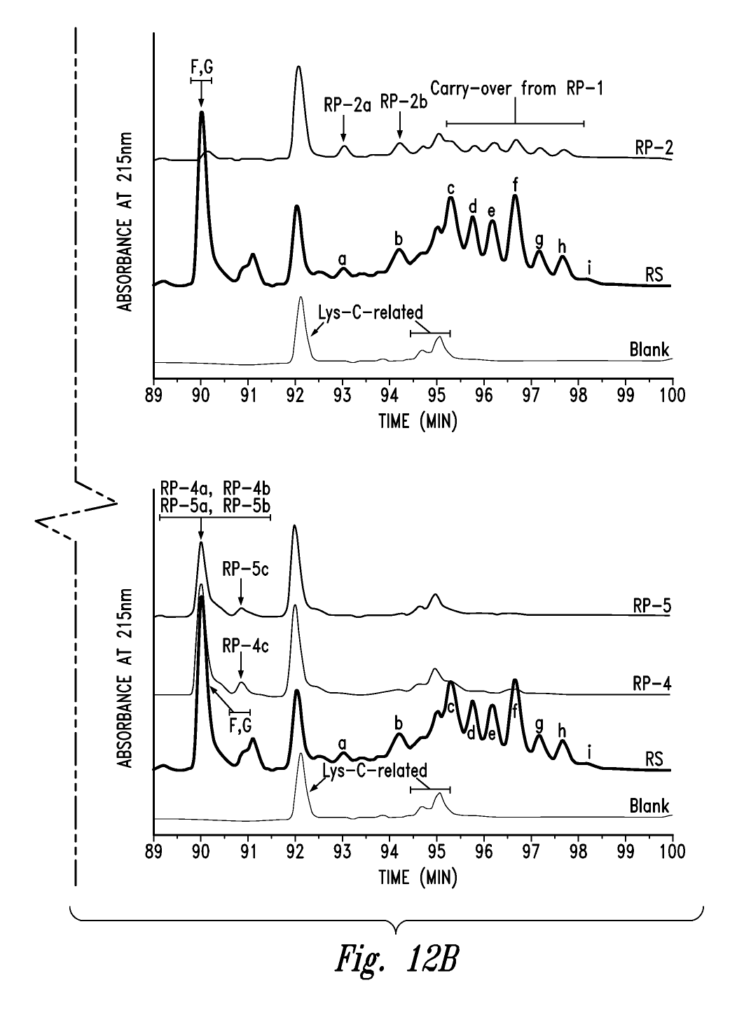

Disulfide variants were isolated by non-reduced RP-HPLC. Five fractions were isolated, as illustrated in FIG. 11. The observed molecular weight, purity and proportional distribution of each fraction are summarized in Table 5. The masses are essentially identical within the capability of the MS instrument, confirming that these species are differentiated by conformation or structural features and not biochemical modification. The purified fractions were employed for further evaluation of nonreduced peptide maps and subsequent analyses.

TABLE 5
Molecular weight, purity, and distribution
of nonreduced RP fractions

| Non-reduced | Observed | Purity of | Proportion |
|---|---|---|---|
| RP-HPLC | Mass | Enriched | Distribution |
| Fraction | (Da) ^{a} | Fraction (%) | (%) |
| RP-1 | 147,357 | 86.3 | 43.1 |
| RP-2 | 147,359 | 58.3 | 8.1 |
| RP-3 | 147,360 | 82.8 | 26.2 |
| RP-4 | 147,361 | 94.2 | 13.6 |
| RP-5 | 147,368 | 97.1 | 9.1 |

^{a} Average masses reported

### Characterization of Disulfide-Linked Peptides in Non-Reduced RP-HPLC Fractions

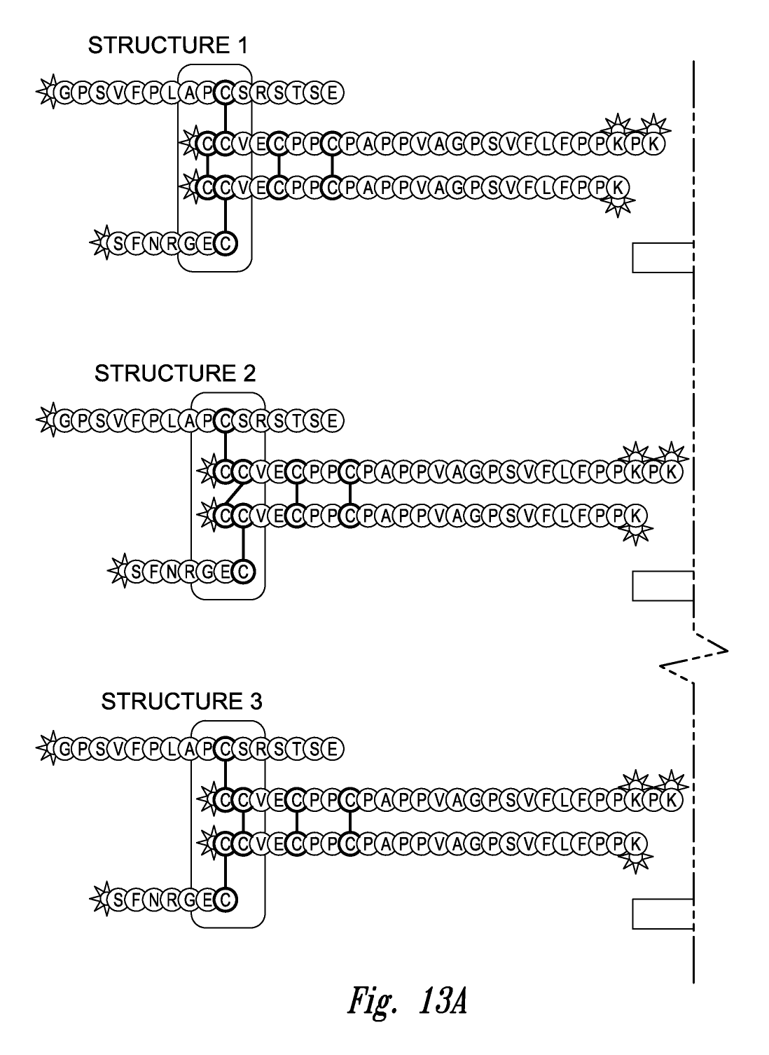

In order to ascertain which disulfide variant eluted at what position in the nonreduced RP-HPLC distribution, enriched fractions (RP-1 through RP-5) were subjected to nonreduced peptide mapping with endoprotease Lys-C. Through this approach the presence of peptides specific to a particular disulfide variant were assessed. Similar profiles throughout most of the chromatograms were obtained from the peptide maps of all RP fractions, including the presence of a subset of the disulfide-containing peptide peaks corresponding to the classical IgG2 structure, peptides A through E (data not shown). This observation indicates that the expected intra-chain disulfide linkages were uniformly present across all fractions. The major differences between the peptide maps were observed in the peak distribution between 90 and 100 min, as illustrated in FIG. 12. Observed masses for the peptides from each fraction are summarized in Table 6, and indicate that each fraction corresponded to a specific group of peptides characteristic of a given variant. Accordingly, fraction RP-1 is characterized by the ˜25 kDa variant (IgG2-B), RP-3 by the ˜15 kDa variant (IgG2-A/B), and RP-4 and RP-5 show only the peptides corresponding to the classical structure (IgG2-A). Notably, RP-2 is characterized by the ˜12 kDa variant, which was not addressed in contemporary reports.

TABLE 6
Distribution of Signature Variant
Peptides for RP Fractions

| | | Observed | |
|---|---|---|---|
| | | Mass | Disulfide |
| Fraction | Peptide | (Da) ^{a} | Variant |
| RP-1 | RP-1a | 26160.85 | IgG2-B |
| | RP-1b | 25547.15 | |
| | RP-1c | 25935.57 | |
| | RP-1d | 25321.88 | |
| | RP-1e | 25709.24 | |
| | | 24794.32 | |
| | RP-1f | 25096.58 | |
| | RP-1g | 24568.39 | |
| RP-2 | RP-2a | 12773.50 | IgG2-C |
| | RP-2b | 12547.61 | |
| RP-3 | RP-3a | 10095.99 | IgG2-A, IgG2-A/B |
| | RP-3b | 9568.29 | |
| | RP-3c | 15450.70 | IgG2-A/B |
| | RP-3d | 15225.48 | |
| | RP-3e | 14999.83 | |
| RP-4 | RP-4a | 5129.09 | IgG2-A |
| | RP-4b | 10096.05 | |
| | RP-4c | 9568.60 | |
| RP-5 | RP-5a | 5129.09 | IgG2-A |
| | RP-5b | 10095.98 | |
| | RP-5c | 9568.36 | |

^{a} Average masses reported

### Determination of Disulfide Connectivity for Each Variant

Unique peptides corresponding of each variant (˜25 kDa, IgG2-B; ˜15 kDa, IgG2-A/B; and ˜12 kDa, IgG2-C) were subjected to secondary digestion with endoprotease Glu-C, and subsequent analysis by LC-MS to confirm the identities of the resulting peptides. In the case of each variant, a peptide of mass 7669 Da was observed following Glu-C digestion, corresponding to peptide H6b (containing Cys-194) coupled to peptide H7-8 (containing Cys-205). This species is identical to that observed from the Glu-C digestion of peptide G from the classic IgG2-A structure, indicating that the linkage pattern of all variants is conserved for the intra-chain C_{H}1 loop. Hence, all differences between the variants must therefore be related to the nature of the linkages between the constituent species—Fab peptide H6a (Cys-136), Fab peptide L12 (Cys-215), and hinge peptide H11 (Cys-224, 225, 228, and 231), as well as the number of copies of each constituent.

In order to determine the specific connectivity of each representative peptide, a novel analytical approach was employed. As previously described, the combination of manual Edman sequencing steps and analysis of the resulting products by LC-MS after each coupling and cleavage cycle affords specific and sequential identification of the connectivity of each disulfide present in non-reduced peptides (Zhang and Cockrill, Anal. Chem. 2009). Fortuitously, the conserved hinge peptide sequence of the IgG2 subclass contains a lysine residue immediately prior to the first hinge cysteine, and hence the N-terminal residue of the H11 hinge peptide is Cys-224. Therefore, the connectivity of disulfide variants can be readily determined by the specific monitoring of leaving and/or residual groups. This approach is illustrated in the following section for the IgG2-A/B (˜15 kDa) variant.

The fundamental concept for this novel approach for connectivity assignment is not dissimilar to the protein ladder sequencing method, in which matrix-assisted laser desorption/ionization mass spectrometry (MALDI-MS) analysis is used to differentiate cleavage products following sequential reactions (Chait et al., Science, 1993 262 89-92; Bartlet-Jones et al., Rapid Commun. Mass Spectrom. 1994 8 737-742). This approach is elegant in that single amino acid residues are sequentially removed without separation, resulting in the presence of a set of nested peptides, each differentiated by the loss of one N-terminal amino acid residue compared to its predecessor. The observed mass difference affords identification of the amino acid residue lost in successive cycles. In theory, this methodology could also be applied to the disulfide-bearing species, as the loss of different leaving groups yields residual groups of specific and characteristic mass. However, the approach is confounded by the inherent nature of MALDI-MS to induce prompt dissociation of disulfide bonds, such that the constituent disulfide-linked peptides are observed in a reduced state (Patterson et al., Anal. Chem. 1994 66 3727-3732; Crimmins et al., Anal. Biochem. 1995 226 355-361; and Qiu et al., Rapid Commun. Mass Spectrom. 2007 21 3520-3525).

### Principle of the Method to Differentiate Structural Variants

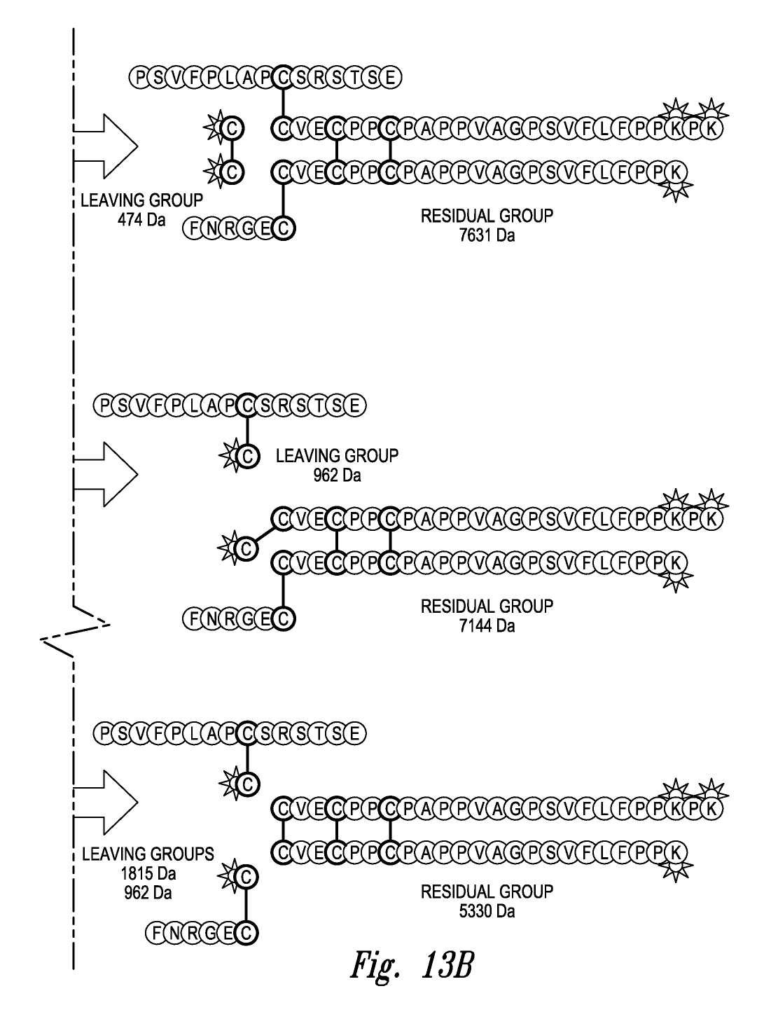

FIG. 13 illustrates three possible structures of the IgG2-A/B variant following secondary digestion with endoprotease Glu-C. These structures are employed to demonstrate the principal of the methodology for determination of disulfide linkage connectivity.

Each of the three structures shown in FIG. 13 contains differing connectivity between the Fab cysteines of H6a and L12 with the first two cysteine residues of the hinge peptides. In each case, the PITC coupling and cleavage cycle results in the generation of “leaving” and “residual” groups that confer elucidation of the disulfide connectivity of the initial non-reduced peptide. Indeed, the technique has the power to identify the presence of mixed populations in spite of the fact that the leaving groups are consistent between variants; for example, the L12 species is common to the leaving groups of both Structures 2 and 3; however the corresponding residual groups for each structure possess unique masses affording detection and discrimination of multiple species simultaneously.

Nonreduced peptides characteristic of each disulfide variant were analyzed through repetitive cycles of Edman-MS in order to ascertain the specific connectivity associated with each, as described in the following sections.

### Characterization of IgG2-C Variant

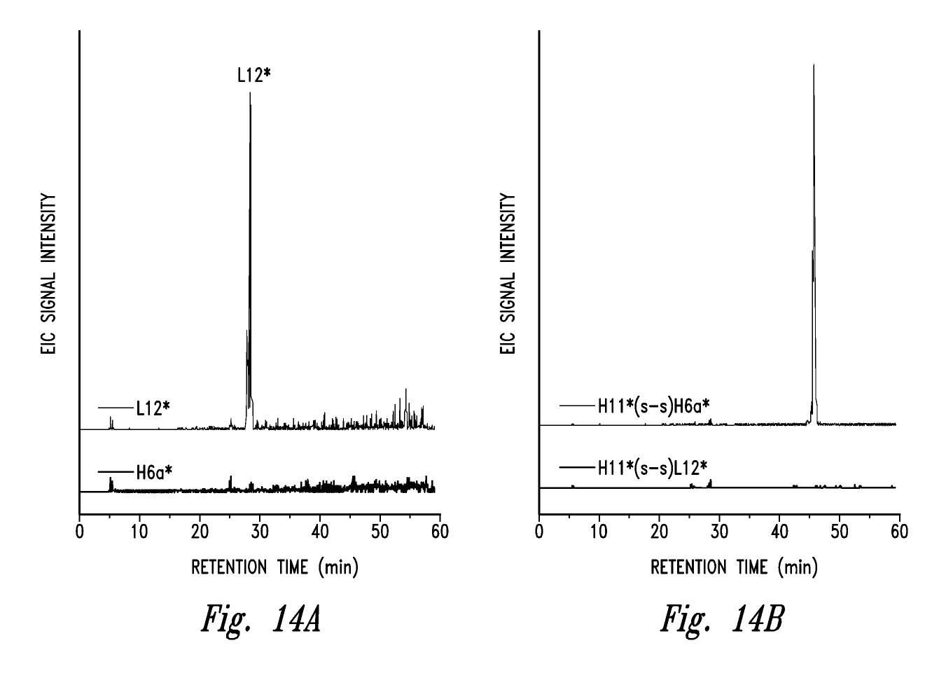

The IgG2-C variant, characterized by the ˜12 kDa nonreduced peptide following digestion with endoprotease Lys-C, contains a single copy of the hinge (H11) and Fab peptides (L12, H6, H7-8). The smaller number of structural elements limits the number of possible arrangements; therefore only two cycles were needed to afford complete elucidation of disulfide connectivity. The EICs for potential leaving and residual groups are shown in FIG. 14.

Examination of the data indicated that only the L12* leaving group was observed, along with the associated residual group consisting of H11*(s-s)H6a* (m/z value 1354.9 for 3+ ion). Note that the number of asterisks in the peptide nomenclature corresponds to the number of cycles and therefore also to the number of amino acids truncated from the corresponding N terminus of the peptide. Therefore L12* is the L12 peptide following a single cycle, with concomitant removal of the native N terminal serine residue. There was no evidence for the H6a* leaving group or the corresponding H11*(s-s)L12* residual group (FIG. 14, first panel). The data indicate that issues of disulfide scrambling are not apparent in this methodology, as previously noted (Zhang and Cockrill, Anal. Chem. 2009).

Interestingly, leaving groups are sometimes characterized by the presence of a doublet of PTH-labeled species eluting in the chromatogram following execution of the conversion step analogous to traditional Edman sequencing, as evidenced for the L12* leaving group (FIG. 14, first panel). Both species in the doublet presented identical masses and fragmentation patterns (data not shown). It is considered that these two species may be the result of racemization during the conversion step to the final PTH derivative (Iida et al., J Chromatogr. A, 1998, 813: 267-275).

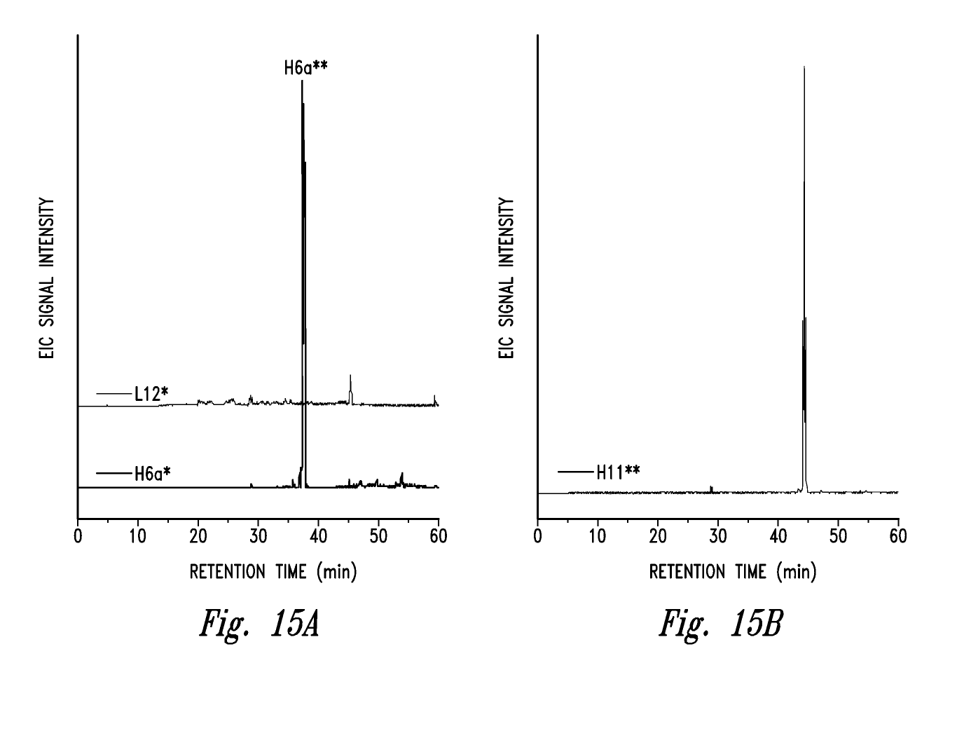

In the second cycle, EICs showed the detection of the H6a** leaving group and the H11** residual group (FIG. 15). No signal was observed for the L12** leaving group (the peak at ˜45 min in FIG. 15, Panel A is an unrelated species, confirmed by MS/MS analysis—data not shown).

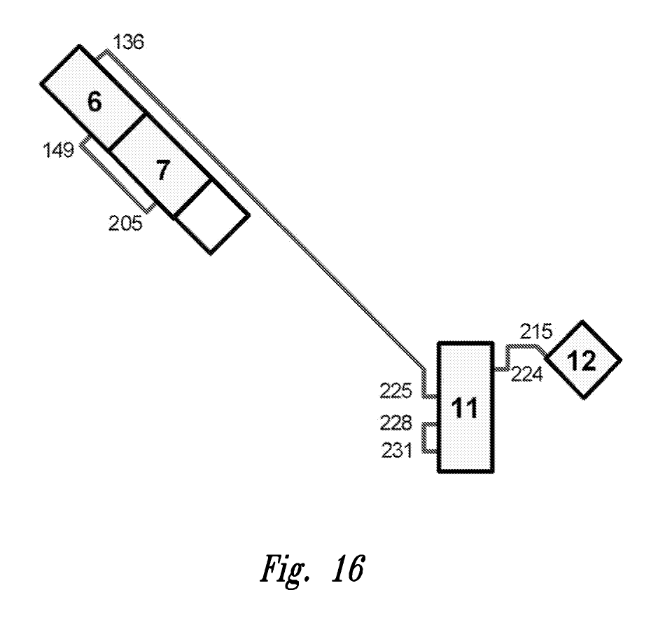

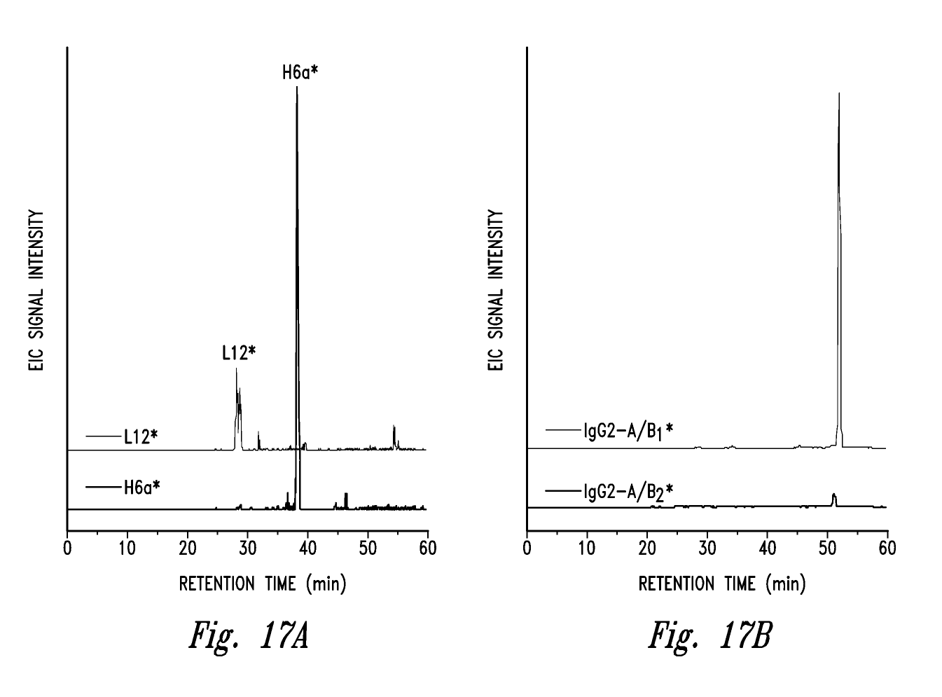

Importantly, the H11** residual group corroborates the precept that the IgG2-C variant contains an intra-chain disulfide linkage in the hinge peptide between Cys-228 and Cys-231. Therefore, the connectivity of the ˜12 kDa peptide characteristic to the IgG2-C disulfide variant is as depicted in FIG. 16.

It is noteworthy that this structure potentially constitutes only one half of the antibody homo-dimer. However, it was previously demonstrated for this rIgG2 molecule that the overall structure is not linked within a half-molecule domain due to the absence of half-molecule species as assessed by SDS-PAGE, but instead must contain connectivity across the symmetrical halves of the antibody homo-dimer structure. Analytical methodology does not exist at this time to determine which constituent species are located on the two sides of the intact IgG2 homo-dimer.

### Characterization of IgG2-A/B Variant

The IgG2A/B variant, characterized by the ˜15 kDa non-reduced peptide following digestion with endoprotease Lys-C, contains two copies of the hinge (H11) peptide and single copy of the Fab peptides (L12, H6, H7-8). As noted previously, the other Fab arm peptide was determined to be consistent with the expected connectivity of peptide G from the classical structure; after secondary digestion of peptide c with endoprotease Glu-C, the connectivity between peptide H6a (Cys-194) and peptide H7-8 (Cys-205) was established. Therefore, the remaining non-reduced species contained two copies of the H11 hinge peptide, and single copies of Fab peptides H6a and L12, which may form several structural variants through differential connectivity of the disulfide linkages, as illustrated in the preceding section discussing the principles of variant differentiation.

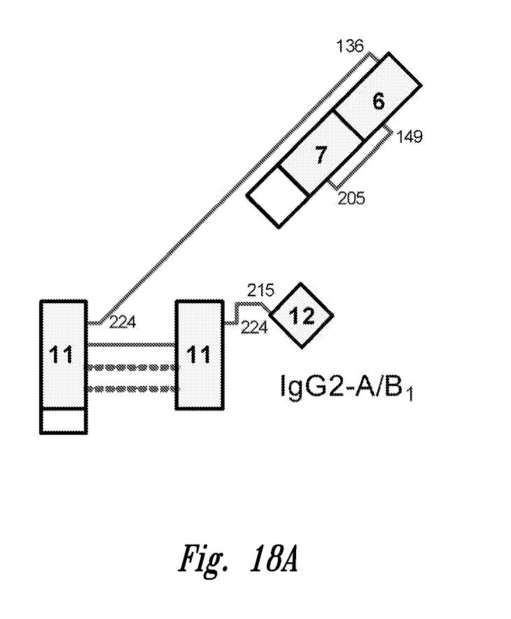

The IgG-2A/B variant was analyzed by repetitive cycles of PITC coupling, cleavage and LC-MS following secondary digestion with endoprotease Glu-C, and the LC-MS data was parsed for masses corresponding to potential leaving and residual groups, noted in FIG. 13. Resulting EICs for leaving and residual groups are presented (FIG. 17).

Of particular note is the fact that signals corresponding to both the L12* and H6a* leaving groups are observed (FIG. 17, first panel). This suggests that either; 1) both L12 and H6a peptides are connected to Cys-224 of the two copies of H11, 2) a mixture of structures is present, or 3) both 1) and 2) are true. Examination of the EICs for the residual groups (FIG. 17, second panel) revealed signal for two different species—H11-12*(s-s)H11* and H6a*(s-s)H11-12*(s-s)H11*(s-s)L12*. This indicates the presence of a mixture of disulfide variants related to the ˜15 kDa species; the first where L12 and H6a are connected to Cys-224 of both H11 peptides (consistent with Structure 3 in FIG. 13), and the second where L12 and H6a are connected to Cys-225 of both H11 peptides (consistent with Structure 1 in FIG. 13). Although not a quantitative analysis, the relative proportions of H6a* and L12*, as well as the various characteristic residual groups, are indicative that Structure 3 corresponds to IgG2-A/B₁, i.e., the predominant variant for IgG2-A/B.

Analysis of the second cycle revealed the presence of a single residual group H11-12**(s-s)H11** (data not shown), confirming that the two structures elucidated from the first cycle data were differentiated by linkage patterns between the L12, H6a, and the first two cysteine residues of the hinge peptide (H11).

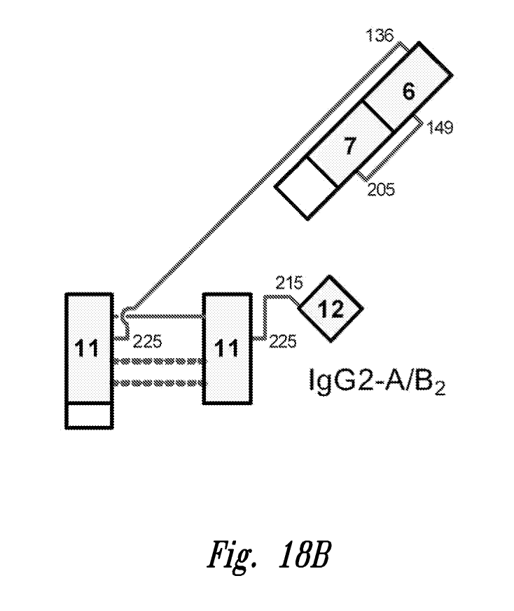

Given that Structure 3 is the predominant variant for IgG2-A/B, then the major leaving group for the second cycle would be the labeled cystine group. This species is considered too small and hydrophilic to be retained by the RP-HPLC conditions used, and therefore was not detected. However, analysis of the second cycle data for the L12** and H6a** leaving group of the minor species (IgG2-A/B₂, Structure 1) did not reveal the presence of these species. This is considered an issue related to the low relative proportion of Structure 1, and the cumulative losses associated with repetitive cycling (Zhang Cockrill, Anal. Chem. 2009). For similar reasons the connectivity between the Cys-228 and Cys-231 residues in the remaining portion of the hinge:hinge dipeptides could not be determined—however it is known that there are interchain linkages in the residual group from the second cycle as this species contained both hinge peptides, obviating intrachain disulfide bonds. The elucidated disulfide structures for IgG2-A/B are depicted in FIG. 18. Note that the dashed lines reflect the expected linkage consistent with the classical hinge structure—however these linkages are not confirmed.

### Characterization of The IgG2-B Variant

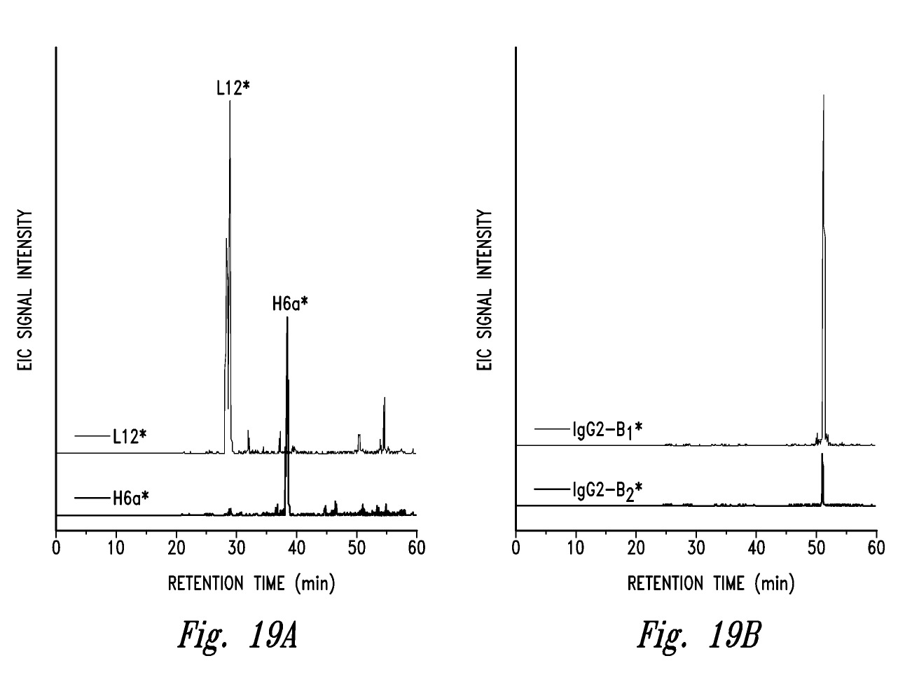

The IgG2-B variant, characterized by the ˜25 kDa non-reduced peptide following digestion with endoprotease Lys-C, contains two copies of the hinge peptide (H11) and two copies of each of the Fab peptides (L12, H6, H7-8). After secondary digestion of peptide f with endoprotease Glu-C, the connectivity between Cys-194 of peptide H6a and Cys-205 of peptide H7-8 was established, consistent with the classical structure. Therefore the remaining non-reduced species containing two copies of the hinge peptide, two copies of H6a and two copies of L12 was analyzed by multiple cycles of PITC coupling, cleavage and LC-MS. The LC-MS data was parsed for masses corresponding to potential leaving and residual groups. Reconstructed EICs of the observed leaving and residual groups from the first cycle are shown (FIG. 19).

Similar to the IgG2-A/B variant, data analysis confirmed the presence of two leaving groups (L12* and H6a*) as well as two residual groups for the IgG2-B variant. The observed residual groups (IgG2-Bᵢ*, mass=8483.6 Da, and IgG2-B₂*, mass=7630.6 Da) correspond to subvariants of IgG2-B; the masses are consistent with species containing either two copies of the hinge peptide (H11* and H11-12*) bound to two copies of H6a* for IgG2-Bᵢ, or two copies of the hinge peptide (H11* and H11-12*) bound to a single copy each of L12* and H6a*. This suggests that the two sub-variant species are differentiated by the connectivity between the Cys-224 of each hinge peptide. For IgG2-Bᵢ the Cys-224 residue on both hinge peptides is connected to Cys-215 of L12 in a symmetrical arrangement, whereas for IgG2-B₂ the residual group is consistent with the loss of single copies of both L12* and H6a*, suggesting that connectivity exists between Cys-224 of one hinge peptide and Cys-205 of L12, and Cys-224 of the other hinge peptide and Cys-136 of H6a. No other residual groups were observed, indicating the absence of other sub-variant species (data not shown).

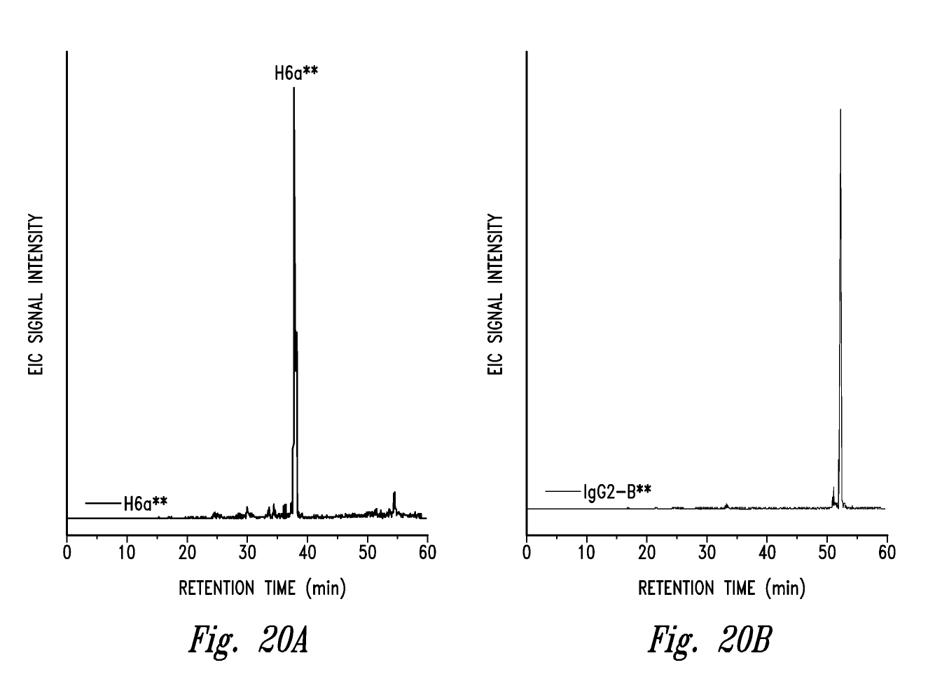

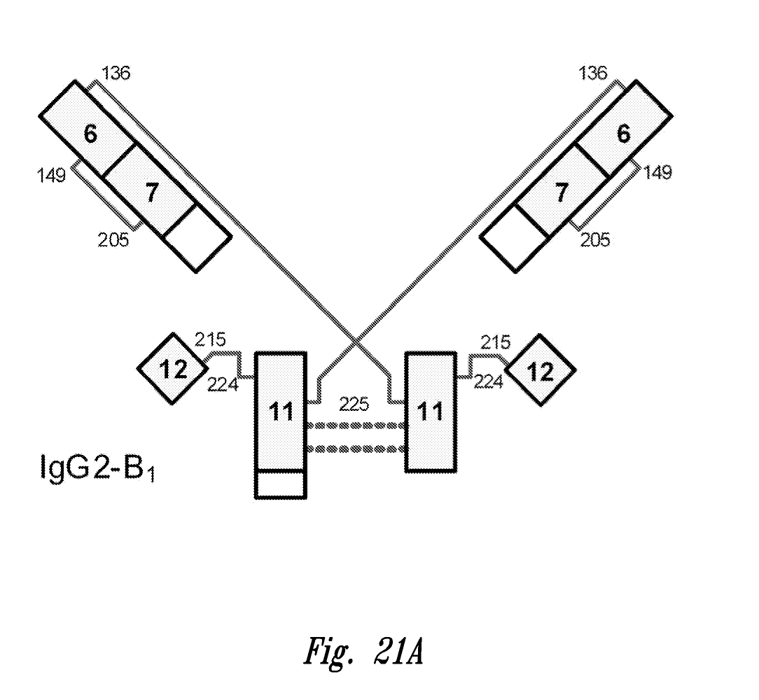

Upon analysis of the two co-eluting residual groups following a second round of coupling and cleavage, it was found that the H6a** group was detected (FIG. 20, first panel). Although no signal was confirmed for the other leaving group (L12**), this was not unexpected due to L12** being characteristic of the minor variant (IgG2-B₂), and the cumulative losses from multiple rounds of analysis. Furthermore, the relatively small size of the L12** species may compromise retention and detection using the RP-HPLC separation. A single residual group was observed (FIG. 20, second panel).

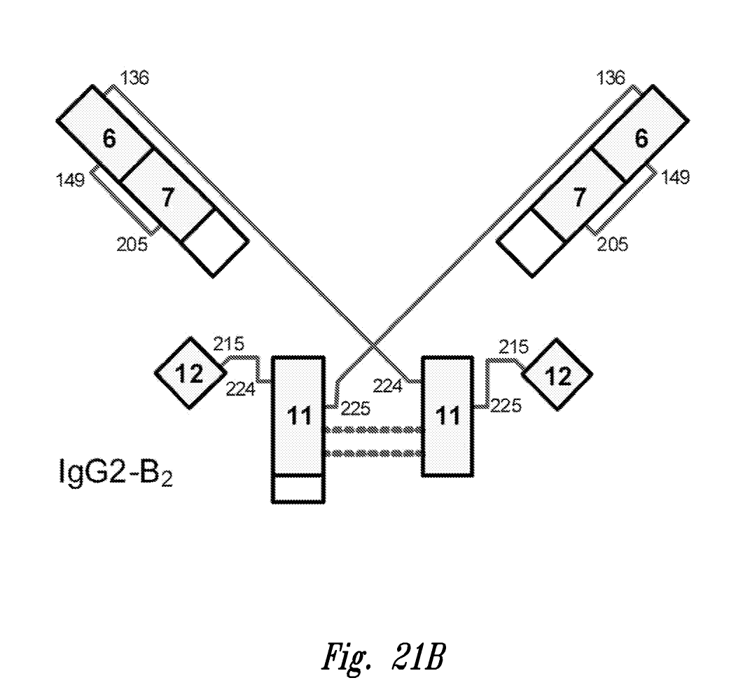

The mass of the residual group (5125.7 Da) corresponds to H11**(s-s)H11-12**, i.e., the dimeric hinge:hinge peptide after loss of Cys-224 and Cys-225 from both peptides. Importantly, this illustrates the linkage between Fab and hinge peptides is limited to the first two cysteine residues only (Cys-224 and Cys-225) on both hinge peptides, and therefore that inter-chain disulfide bonds exist between the two hinge peptides. Given the previously identified limitations of this technique for the cycling efficiency, data could not be obtained to ascertain the geometry of the linkages between Cys-228 and Cys-231 of each hinge peptide—however it is known that there are interchain linkages in the residual group from the second cycle as this species contained both hinge peptides, obviating intrachain disulfide bonds (Zhang and Cockrill, Anal. Chem. 2009). Hence, overall the IgG2-B variant contains two distinct disulfide sub-structures denoted IgG2-Bᵢ and IgG-2B₂ as illustrated in FIG. 21. Note that the dashed lines reflect the expected linkage consistent with the classical hinge structure—however these linkages are not confirmed.

### CONCLUSIONS

These data elucidate previously uncharacterized disulfide connectivities between Fab and hinge peptides among recently reported disulfide variants of recombinant IgG2 molecules (Wypych et al., J. Biol. Chem. 2008, 283, 16194-16205). The analytical methodology utilized was a newly developed technique involving the coupling of traditional Edman N-terminal sequencing chemistry and LC-MS analysis of sequential cycling products (Zhang et al., Anal. Chem., 2009). Prior analysis of recombinant insulin as a well characterized test protein demonstrated that this powerful technique affords facile analysis and data interpretation, obviates the occurrence of disulfide scrambling, and therefore affords a comprehensive assessment of the architecture of complex disulfide-linked peptides.

Of the four IgG2 disulfide variants identified (IgG2-A or classical, IgG2-A/B, IgG2-B, and IgG2-C), two of the populations demonstrated the presence of multiple linkage geometries between the hinge and Fab regions (i.e., IgG2-A/Bᵢ and IgG2-A/B₂; IgG2-Bᵢ and IgG2-B₂). The proportions of each variant (Table 1) demonstrated that the classical IgG2-A structure was not the predominant species for the studied rIgG2 antibody, consistent with earlier reports (Wypych et al., J. Biol. Chem., 2008, 283, 16194-16205; Martinez et al., Biochemistry, 2008, 47, 7496-7508).

For IgG2-B, the two variants differ through the symmetry of the disulfide linkages. In IgG2-B1, the predominant species, the two copies of the L12 peptides are connected through their cysteines (Cys-215) to the first cysteine (Cys-224) of each hinge peptide, H11. The H6 cysteine (Cys-136) on both “sides” of the molecule is connected to the second hinge cysteine (Cys-225), resulting in a symmetrical arrangement. In the minor subvariant, IgG2-B2, this symmetry is absent—one L12 peptide is bound through its cysteine residue (Cys-215) to the first cysteine (Cys-224) on one side of the hinge:hinge dipeptide, whereas the H6 cysteine (Cys-136) is connected to the first cysteine (Cys-224) on the other hinge peptide, H11. Similarly, the second hinge cysteine residues (Cys-225) are linked to both remaining L12 and H6. These linkage patterns are illustrated in FIG. 13.

In the case of IgG2-A/Bᵢ, the predominant species of the IgG2-A/B variant, linkages were elucidated between the light chain L12 peptide (Cys-215) and the first cysteine residue (Cys-224) of one heavy chain hinge peptide, H11. Concomitantly linkage between heavy chain H6 peptide (Cys-136) and the first cysteine residue (Cys-224) of the other heavy chain hinge peptide was determined. For the minor subvariant, IgG2-A/B₂, the linkage between L12 and H6 Fab peptides was determined to be to the second cysteine residue (Cys-225) of the H11 hinge peptides. These structures are depicted in FIG. 14.

IgG2-C is a newly reported isoform that is similar to IgG2-B in the proportions of its constituent peptides (Fab and hinge in 1:1 proportions, unlike IgG2-A/B which has a 1:2 ratio). The differentiating factor for IgG2-C vs IgG2-B is that the former demonstrated linkage homogeneity—only a single variant was detected. In IgG2-C the L12 cysteine (Cys-215) is coupled to the first hinge cysteine (Cys-224), and the H6 cysteine (Cys-136) is linked to the second hinge cysteine (Cys-225). The absence of dimeric hinge:hinge structures in the IgG2-C variant demonstrate the presence of intra-chain linkage between the remaining hinge cysteines (Cys-228 and Cys-231). However, as previously noted, cross-domain linkages must exist as the presence of half-molecule species was not observed. The bonding structure for IgG2-C is presented in FIG. 15.

Detailed evaluation of the classical IgG2-A structure (depicted in FIG. 16) was not undertaken beyond confirmation of the connectivity of peptides A through G in the nonreduced Lys-C peptide map of unfractionated material; the presence of multiple nonreduced RP-HPLC fractions bearing the same apparent disulfide linkages (RP-4 and RP-5) suggests possible subtle differences between the two. Whether this is related to differential bonding in the hinge:hinge dipeptide (i.e., parallel “ladder” vs. “crossed” linkages) or some other conformational disparity remains to be investigated.

As noted in contemporary literature, the immunoglobulin G subclasses possess demonstrable differences in their physicochemical (Dillon et al., J. Biol. Chem. 2008, 283, 16206-16215; Roux et al., J. Immunol 1997 159 3372-3382) and biological properties (Jeffries et al., Immunol Lett. 2002 82 57-65). Furthermore, it has been reported that the IgG2 structural variants may potentially have discernable differences in their characteristic properties (Dillon et al., J. Biol. Chem. 2008, 283, 16206-16215).

The observation of multiple subvariants for particular IgG2 disulfide isoforms demonstrate the degree of characterization required for full characterization of a biotherapeutic product. Of interest for future evaluation will be the influence of cell line and culture conditions on the distribution of the subvariant population, or potentially even the presence of alternative disulfide architecture in recombinant IgG2s. Certainly ongoing investigations have demonstrated the ability to exert a degree of control over the heterogeneity of IgG2 disulfide variants through site-directed mutagenesis involving Cys→Ser mutations (Allen et al., Biochemistry 2009 48 3755-3766). At present the implication of the presence of multiple subvariants with respect to biological properties remains to be fully understood.

While the compositions and methods of this invention have been described in terms of some embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The references cited herein throughout, to the extent that they provide exemplary procedural or other details supplementary to those set forth herein, are all specifically incorporated herein by reference.

What is claimed:

1. A method of determining the disulfide linkage connectivity in a polypeptide comprising incubating the polypeptide with a reagent to cap existing free sulfhydryl, dividing the polypeptide into fragments containing disulfide bonds, separating and collecting the fragments, removing sequentially the amino acid residues from the fragments, and identifying the position and connectivity of the disulfide linkages between cysteine residues.

2. The method of claim 1 wherein the polypeptide is first subjected to denaturation or unfolding.

3. The method of claim 2 wherein the denaturation and/or unfolding is achieved by chemical or mechanical means.

4. The method of claim 3 where the chemical cleavage is performed by a method selected from a group consisting of Cyanogen bromide (CNBr), BNPS-skatole, formic acid, hydroxylamine, and 2-nitro-5-thiocyanobenzoic acid (NTCB).

5. The method of claim 1 wherein the free sulfhydryl groups are capped with a chemical reagent.

6. The method of claim 5 wherein the chemical reagent is an alkylating reagent.

7. The method of claim 6 wherein the alkylating reagent is N-ethylmaleimide.

8. The method claim 1 wherein the polypeptide is divided into fragments by chemical, enzymatic or proteolytic means.

9. The method claim 8 wherein the enzyme or protease is selected from a group consisting of trypsin, Lys-C, and Glu-C.

10. The method of claim 1 wherein multiple rounds of division of the polypeptide are employed, utilizing different enzymes or proteases in any given order.

11. The method of claim 1 further comprising after the dividing the polypeptide into fragments, secondarily dividing the fragments.

12. The method of claim 1 wherein the separation of fragments is performed by electrophoretic or chromatographic means.

13. The method of claim 11 wherein the separation of fragments is performed by reverse-phase high performance liquid chromatography (RP HPLC).

14. The method of claim 12 wherein the separated fragments are further subdivided by chemical or enzymatic or proteolytic means.

15. The method claim 13 wherein the enzyme or protease is selected from a group consisting of trypsin, Lys-C, and Glu-C.

16. The method of claim 1 wherein the separated fragments are treated with a reagent to facilitate sequential removal of N-terminal amino acid residues.

17. The method of claim 16 wherein the reagent is phenylisothiocyanate (PITC).

18. The method of claim 1 or 14 wherein at least one N-terminal amino acid residue from the separated fragments is removed.

19. The method of claim 18 wherein the removal of N-terminal amino acid residue(s) is achieved by acid hydrolysis.

20. The method of claim 19 wherein the acid hydrolysis is achieved using anhydrous trifluoroacetic acid (TFA).

21. The method of claim 1 wherein leaving and residual groups are separated after removal of the N-terminal amino acid residues from the separated fragments.

22. The method of claim 21 wherein the separation of leaving and residual groups is achieved by electrophoretic or chromatographic means.

23. The method of claim 22 wherein the separation of leaving and residual groups is performed by reverse-phase high performance liquid chromatography (RP HPLC).

24. The method of claims 21, 22, and 23 wherein the identity of leaving and residual groups is confirmed by mass spectrometry.

25. A method of determining the disulfide linkage connectivity in a polypeptide wherein recovery of residual groups and removal of subsequent N-terminal amino acid is performed with successive iterations of claim 24.
